(19) United States
(12) Patent Application Publication
(10) Pub. No.: US 2024/0407655 A1
(43) Pub. Date: Dec. 12, 2024

(54) INTEGRATED PRESSURE DIAPHRAGM

(71) Applicant: EDWARDS LIFESCIENCES CORPORATION, Irvine, CA (US)

(72) Inventors: Alexander H. Siemons, Yorba Linda, CA (US); Gustavo Rios, Pasadena, CA (US)

(21) Appl. No.: 18/811,187

(22) Filed: Aug. 21, 2024

Related U.S. Application Data
(63) Continuation of application No. PCT/US2023/015423, filed on Mar. 16, 2023.
(60) Provisional application No. 63/321,556, filed on Mar. 18, 2022.

(51) Int. Cl. A61B 5/021; A61B 5/00

(52) U.S. Cl. CPC A61B 5/02141; A61B 5/686; A61B 5/6869; A61B 2560/045; A61B 2560/0462; A61B 2562/0247; A61B 2562/12; A61B 2562/162

(57) ABSTRACT

An implantable sensor device includes a cylindrical housing, a deflectable diaphragm associated with a sidewall of the housing, and a pressure sensor device housed within the housing. A transduction medium is disposed within the housing over at least a portion of the pressure sensor device.

### RELATED APPLICATION

This application is a continuation of International Patent Application No. PCT/US23/15423, filed Mar. 16, 2023 and entitled INTEGRATED PRESSURE DIAPHRAGM, which claims priority to U.S. Provisional Patent Application Ser. No. 63/321,556, filed on Mar. 18, 2022 and entitled INTEGRATED PRESSURE DIAPHRAGM, the complete disclosures of which is hereby incorporated by reference in their entirety.

### BACKGROUND

The present disclosure generally relates to the field of sensor devices. Some sensor devices, such as those suitable for medical implantation, can include deflectable diaphragms. Mechanical sealing between such diaphragms and the associated sensor housing/structure can affect suitability of such devices for certain applications.

### SUMMARY

Described herein are methods, systems, and devices that facilitate integration of sensor diaphragms with sensor housing/enclosure structures and/or layers. In particular, various pressure sensor packaging solutions are disclosed that provide for deposition of layer(s) of metal or other material over substrate structure(s) to form integrated diaphragm layer(s). In some examples, diaphragms of inventive sensor devices disclosed herein are advantageously transverse-facing.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates certain human anatomy showing example sensor implant locations in accordance with one or more examples.

FIG. 2 is a block diagram representing a system for wirelessly monitoring one or more physiological parameters associated with a patient according to one or more examples.

FIG. 3A is a side view of a piezoresistive pressure sensor device in accordance with one or more examples.

FIG. 3B is a side view of the piezoresistive pressure sensor of FIG. 3A, wherein a diaphragm of the sensor is deflected in accordance with one or more examples.

FIG. 4A is a side view of a capacitive pressure sensor device in accordance with one or more examples.

FIG. 4B is a side view of the capacitive pressure sensor of FIG. 4A, wherein a diaphragm of the sensor is deflected in accordance with one or more examples.

FIG. 5A shows a front and side perspective view of a sensor implant device in accordance with one or more examples.

FIG. 5B shows an exploded view of the sensor can package of the sensor implant device of FIG. 5A in accordance with one or more examples.

FIGS. 6A and 6B show cross-sectional views of a distal portion of a sensor implant device in accordance with one or more examples.

FIG. 7 is a graph showing relationships between sensor diaphragm thickness, surface area, and sensitivity in accordance with one or more examples.

FIG. 8 is a block diagram showing a deposition system in accordance with one or more examples.

FIGS. 9A, 9B, and 9C show perspective, longitudinal cross-sectional, and axial cross-sectional views, respectively, of a sensor device having an integrated diaphragm in accordance with one or more examples.

FIG. 9D shows a sensor device having a dimple-type diaphragm form in accordance with one or more examples.

FIGS. 10A, 10B, and 10C provide a flow diagram illustrating a process for packaging a sensor device in accordance with one or more examples.

FIGS. 11A, 11B, and 11C provide images of pressure sensor packaging components corresponding to operations of the process of FIGS. 10A, 10B, and 10C according to one or more examples.

FIG. 12 shows an axial cross-sectional view of a sensor device comprising more than two circumferentially-distributed diaphragms in accordance with one or more examples.

FIG. 13 shows a side view of a sensor device comprising a plurality of axially-offset diaphragms in accordance with one or more examples.

FIG. 14 shows a perspective view of a sensor device having axially- and/or circumferentially-offset diaphragms.

FIG. 15A shows a perspective view of a sensor device having an integrated axial diaphragm in accordance with one or more examples.

FIG. 15B shows a perspective view of a sensor device having an integrated axial diaphragm in accordance with one or more examples.

FIG. 16 is a cutaway view of a human heart and associated vasculature showing certain catheter access paths for sensor device implantation procedures in accordance with one or more examples.

FIGS. 17A, 17B. 17C, and 17D provide a flow diagram illustrating a process for packaging a sensor device in accordance with one or more examples.

FIGS. 18A, 18B, 18C, 18D, 18E, 18F, and 18G provide images of pressure sensor packaging components corresponding to operations of the process of FIGS. 17A, 17B, 17C, and 17D according to one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to various examples. Although certain spatially relative terms, such as “outer,” “inner,” “upper,” “lower,” “below,” “above.” “vertical,” “horizontal,” “top,” “bottom,” “distal,” “proximal,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

The present disclosure relates to systems, devices, and methods for packaging devices configured for sensing and/or telemetric monitoring of one or more physiological parameters of a patient (e.g., blood pressure). Such pressure sensing/monitoring may be performed using cardiac implant devices having integrated pressure sensor diaphragms and/or associated components. Such devices can advantageously be packaged for long-term implantation in the cardiac environment. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

As described in detail below, implantable pressure sensors can be used to measure pressure levels in various conduits and chambers of body, such as in the various chambers of the heart. However, due to the accessibility and environmental conditions typically associated with the conduits/chambers of the heart and/or other potential sensor implant locations within a patient, only certain types of sensors and sensor packagings may be suitable for implantation for a given application. Examples of the present disclosure relate to the packaging of pressure sensor implant devices including certain electronics and telemetry features to allow for data and/or power communication wirelessly between the implanted sensor devices and one or more devices or systems external to the patient.

Aspects of the present disclosure relate to sensor devices, such as wireless implantable pressure sensor devices and other devices comprising deflectable diaphragm components. In particular, inventive features disclosed herein can be implemented in the context of implantable sensor devices, wherein integrated diaphragm features can advantageously provide biocompatible sealing and/or encapsulation of internal sensor components, such as pressure-transduction media, circuitry, and/or structural components of the device.

With respect to implantable pressure sensor devices, anatomical considerations can necessitate the use of sensor devices having relatively small form factors. For example, it may be desirable to implant sensor devices, such as pressure sensor devices, using transcatheter procedures, wherein the sensor device is advanced to the target implantation site through one or more venous or arterial blood vessels and/or various tortuous access paths. Examples of the present disclosure advantageously can be implemented in sensor devices having a sufficiently small profile/size to be transported by and/or within a catheter, sheath, or other instrument configured for transcatheter access/use. In addition to sizing constraints associated with implantable sensor devices (e.g., pressure sensor devices), sensor sensitivity and/or dynamic range requirements or desires likewise may drive sensor design. For example, with respect to pressure sensor devices, deflectable pressure diaphragms associated with such devices may be designed in a manner as to provide sufficient sensitivity to pressure conditions to which the device is exposed.

Furthermore, implantable sensor devices, such as diaphragm-equipped pressure sensor devices, may further need to provide biocompatibility and/or encapsulation characteristics suitable for in vivo implantation. For example, with respect to implantation within certain anatomy, such as within a chamber of a heart, or other fluid-filled anatomical vessel/chamber, such environments can present certain pressure, turbulence, and corrosion conditions, which may be associated with fluid/blood characteristics and/or cardiac cycling. Relative to non-implant environments, the human body represents a very harsh environment for electrical implant devices. Examples of the present disclosure provide sensor implant devices that provide extended-duration and/or lifetime hermetic seals/sealing, which can be advantageous and/or critical for various reasons. For example, such hermetic sealing can prevent components of the environmental blood from degrading or otherwise interfering with the sensor and associated electronics. In addition, hermetic sealing of examples of the present disclosure can help prevent any non-biocompatible components of or associated with the sensor implant device from creating/causing toxic conditions within the body. Examples of the present disclosure furthermore provide sensor implant devices with ion-deposited shell components that are relatively thin and provide hermitically-scaled, integrated sensor diaphragms. Such designs can have a reduced package thickness compared to certain other welded-metal solutions, which may include interfaces between metal and/or ceramic components thereof that occupy undesirable amounts of space. By integrating multiple functional components into a single thin shell, the total package thickness/size can be minimized.

Certain examples are disclosed herein in the context of cardiac implant devices. However, although certain principles disclosed herein may be particularly applicable to the anatomy of the heart, it should be understood that sensor implant devices in accordance with the present disclosure may be implanted in, or configured for implantation in, any suitable or desirable anatomy. Furthermore, examples of the present disclosure may be utilized in non-biological environments as well.

The anatomy of the heart is described below to assist in the understanding of certain inventive concepts disclosed herein. In humans and other vertebrate animals, the heart generally comprises a muscular organ having four pumping chambers, wherein the blood flow therein is at least partially controlled by various heart valves, namely, the aortic, mitral (or bicuspid), tricuspid, and pulmonary valves. The valves may be configured to open and close in response to pressure gradients present during various stages of the cardiac cycle (e.g., relaxation and contraction) to control the flow of blood to respective regions of the heart and/or to blood vessels (e.g., pulmonary, aorta, etc.). The contraction of the various heart muscles may be prompted by signals generated by the electrical system of the heart, which is discussed in detail below.

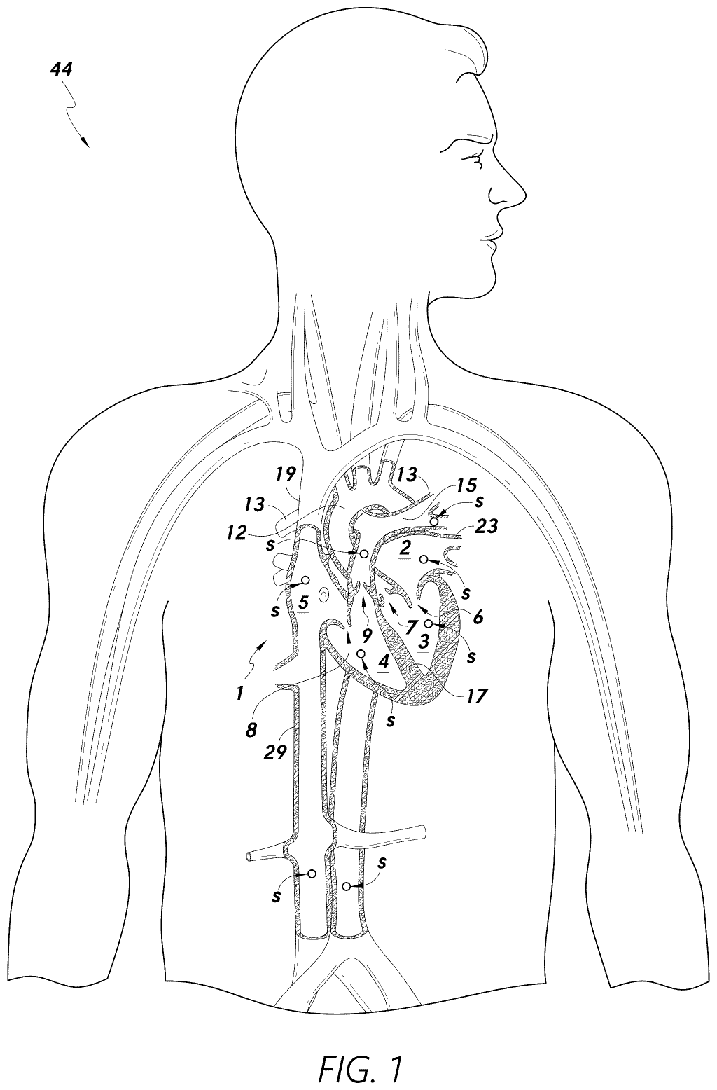

FIG. 1 illustrates an example representation of a heart 1 and associated anatomy having various features relevant to certain examples of the present inventive disclosure. The illustrated anatomy shows example implant locations for sensor devices in accordance with aspects of the present disclosure. Generally, the heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11.

The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. Therefore, direct or indirect measurement/monitoring of pressure and/or other parameter(s) using implant devices can provide better outcomes than purely observation-based solutions. For example, without direct or indirect monitoring of cardiac pressure, it can be difficult to infer, determine, or predict the presence or occurrence of congestive heart failure or other pathologies. Treatments or approaches not involving direct or indirect pressure monitoring may involve measuring or observing other present physiological conditions of the patient, such as measuring body weight, thoracic impedance, right heart catheterization, or the like.

### Cardiac Pressure Monitoring

Cardiac pressure monitoring in accordance with examples of the present disclosure may provide a proactive intervention mechanism for preventing or treating congestive heart failure. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with examples of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to examples of the present disclosure, normal ventricular filling pressures may advantageously be maintained, thereby preventing or reducing effects of heart failure, such as dyspnea.

Pressure sensor devices disclosed herein may be implanted in any of the chambers/vessels of the heart or other blood vessels (e.g., aorta, vena cava). FIG. 1 shows a number of example implantation sites for implantable sensor devices (denoted as ‘s’ in FIG. 1) in accordance with aspects of the present disclosure. For example, as shown, sensor implant devices having integrated diaphragm components in accordance with the present disclosure can be implanted in the right atrium, right ventricle, left atrium, left ventricle, pulmonary arteries, inferior vena cava, aorta, or other anatomy. Generally, pressure elevation in the left atrium may be particularly correlated with heart failure, and so implantation of sensor implant devices in the left atrium may be desirable in some cases.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension can affect the reliability of pulmonary artery pressure measurement for estimating left-sided filling pressure. Therefore, pulmonary artery pressure measurement alone may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment. The present disclosure provides systems, devices, and methods for packaging implantable pressure sensors configured to provide direct measurements of pressure conditions at the implantation site.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values, but rather estimated information, which may not provide the requisite specificity in some cases. Furthermore, as ultrasound, or similar, imaging equipment is typically not found in the home environment, nor are typical patients competent to use such equipment, ambulatory devices in accordance with aspects of the present disclosure that are relatively easy to operate may be desirable in certain situations.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with examples of the present disclosure may be used to predict heart failure up two weeks or more before the manifestation of symptoms or markers of heart failure (e.g., dyspnea). When heart failure predictors are recognized using cardiac pressure sensor examples in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

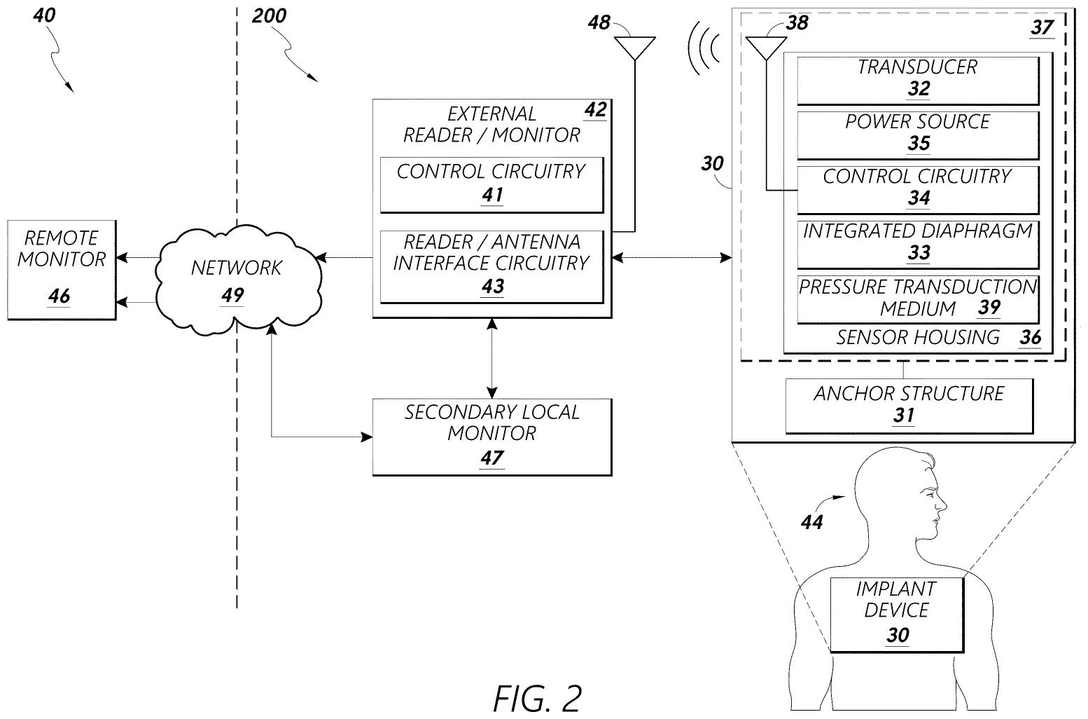

FIG. 2 is a block diagram representing a system 200 for wirelessly monitoring one or more physiological parameters associated with a patient according to one or more examples. FIG. 2 shows an implant device 30 comprising a sensor device 37, which may have associated therewith certain anchoring structure 31. For example, the anchoring structure 31 may be configured to anchor in and/or to one or more biological tissue walls. Although various examples of implantable sensor devices are illustrated and described in the present disclosure without separate anchoring structure, it should be understood that such omissions are solely for the purpose of clarity and any of the examples disclosed herein may have associated therewith certain anchoring structure for anchoring the device to biological tissue/anatomy at the implantation site.

The sensor device 37 may be a pressure sensor according to any of the examples disclosed herein. In some examples, the sensor 37 comprises a transducer 32, such as a MEMS pressure transducer, as well as certain control circuitry 34, which may be embodied in, for example, an application-specific integrated circuit (ASIC) and/or one or more passive devices (e.g., resistors, capacitors, inductors, etc.). The sensor device 37 further includes an integrated diaphragm 33, which is integrated as a unitary layer(s) with at least a portion of the sensor housing 36. In some examples, the sensor housing 36 includes a radio-frequency-transparent base housing, which houses at least a portion of the antenna 38, as well as a transducer-enclosing shell, wherein the shell comprises at least one unitary layer including sidewall and/or endcap wall(s) as well as the diaphragm 33 formed therein. Further details regarding such features are presented in detail below. A pressure transduction medium 39, such as oil, gel, epoxy, or another medium, is disposed within the housing 36 over the transducer 32 in a manner as to translate deflection of the diaphragm 33 into pressure against the transducer 32 to allow for monitoring of such pressure.

The control circuitry 34 of the sensor device 37 may be configured to process signals received from the transducer 32 and/or communicate signals associated there with wirelessly through biological tissue using the antenna 38. The antenna 38 may comprise one or more coils or loops of conductive material, such as copper wire or the like, or piezoelectric resonator(s), or other wireless signal transmission component(s). In some examples, at least a portion of the transducer 32, control circuitry 34, and/or the antenna 38 are at least partially disposed or contained within the sensor housing/packaging 36 structure, which may comprise any type of material and may advantageously be at least partially hermetically sealed. The housing 36, as well as the diaphragm 33, may be formed at least in part using vapor deposition, as described in greater detail below.

The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The housing/packaging 36 may comprise one or more tubes, cans, substrates/boards or other structures comprising glass or other rigid material(s) in some examples, which may provide mechanical stability and/or protection for the components housed therein. In some examples, the housing/packaging 36 is at least partially flexible. For example, the housing/packaging may comprise polymer or other flexible structure/material, which may advantageously allow for folding, bending, or collapsing of aspects of the sensor 37 to allow for passage thereof through a catheter or other introducing means.

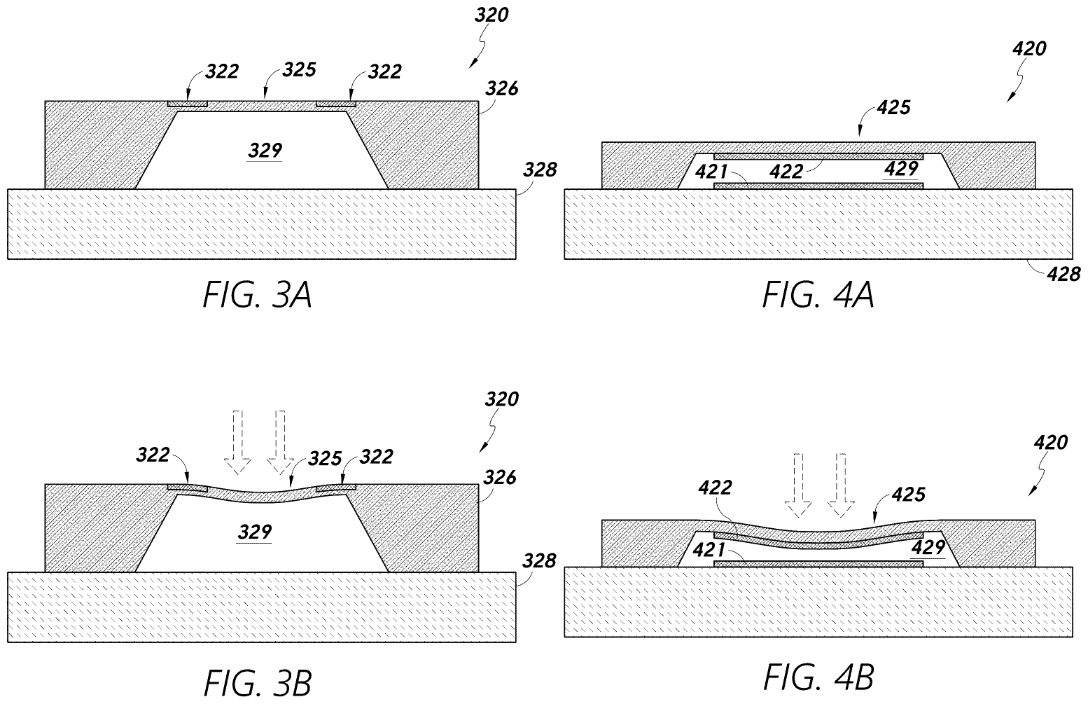

The transducer 32 may comprise any type of sensor means or mechanism. For example, the transducer 32 may be a force-collector-type pressure sensor. In some examples, the transducer 32 comprises a diaphragm, piston, bourdon tube, bellows, or other strain- or deflection-measuring component(s) to measure strain or deflection applied over an area/surface thereof. The transducer 32 may be associated with the housing/packaging 36, such that at least a portion thereof is contained within or attached to the housing/packaging 36. In some examples, the transducer 32 comprises or is a component of a piezoresistive MEMS pressure sensor, which may be configured to use bonded or formed conductors to detect strain due to applied pressure, wherein resistance increases as pressure deforms the component/material, as described below in connection with FIGS. 3A and 3B. Alternatively, the transducer may comprise or be a component of a capacitive pressure sensor, as described below in connection with FIGS. 4A and 4B. The transducer 32 may incorporate any type of material, including but not limited to silicon (e.g., monocrystalline), polysilicon thin film, bonded metal foil, thick film, silicon-on-sapphire, sputtered thin film, and/or the like.

In some examples, the transducer 32 comprises or is a component of an electromagnetic pressure sensor, which may be configured to measure the displacement of a diaphragm by means of changes in capacitance, linear variable displacement transducer (LVDT) functionality, Hall Effect, or eddy current sensing. In some examples, the transducer 32 comprises or is a component of a piezoelectric strain sensor. For example, such a sensor may determine strain (e.g., pressure) on a sensing mechanism based on the piezoelectric effect in certain materials, such as quartz.

In some examples, the transducer 32 comprises or is a component of a strain gauge. For example, a strain gauge example may comprise a pressure sensitive element on or associated with an exposed surface of the transducer 32. In some examples, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

The transducer 32 can comprise one or more MEMS pressure sensor devices, as described in detail herein, mounted in or to a board, or the like. Furthermore, the transducer 32 may be covered in one or more layers of transduction medium 39 and/or vapor-deposited, biocompatible material, as described in detail below. In some examples, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In the system 200 of FIG. 2, the implant device 30 is implanted in a patient 44 for the purpose of monitoring one or more physiological parameters (e.g., left atrial pressure). The patient 44 can have the medical implant device 30 implanted in, for example, his/her heart (not shown), or associated physiology. For example, the implant device 30 can be implanted at least partially within the left atrium of the patient's heart.

In certain examples, the monitoring system 200 can comprise at least two subsystems, including the implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antenna coils). The monitoring system 200 can further include an external (e.g., non-implantable) subsystem that includes an external reader 42 (e.g., coil), which may include a wireless transceiver that is electrically and/or communicatively coupled to certain control circuitry 41. In certain examples, both the internal and external subsystems include a corresponding coil antenna for wireless communication and/or power delivery through patient tissue disposed therebetween. The sensor implant device 30 can be any type of implant device. In some examples, the implant device 30 comprises a pressure sensor integrated with another functional implant structure, such as a prosthetic shunt or stent device/structure.

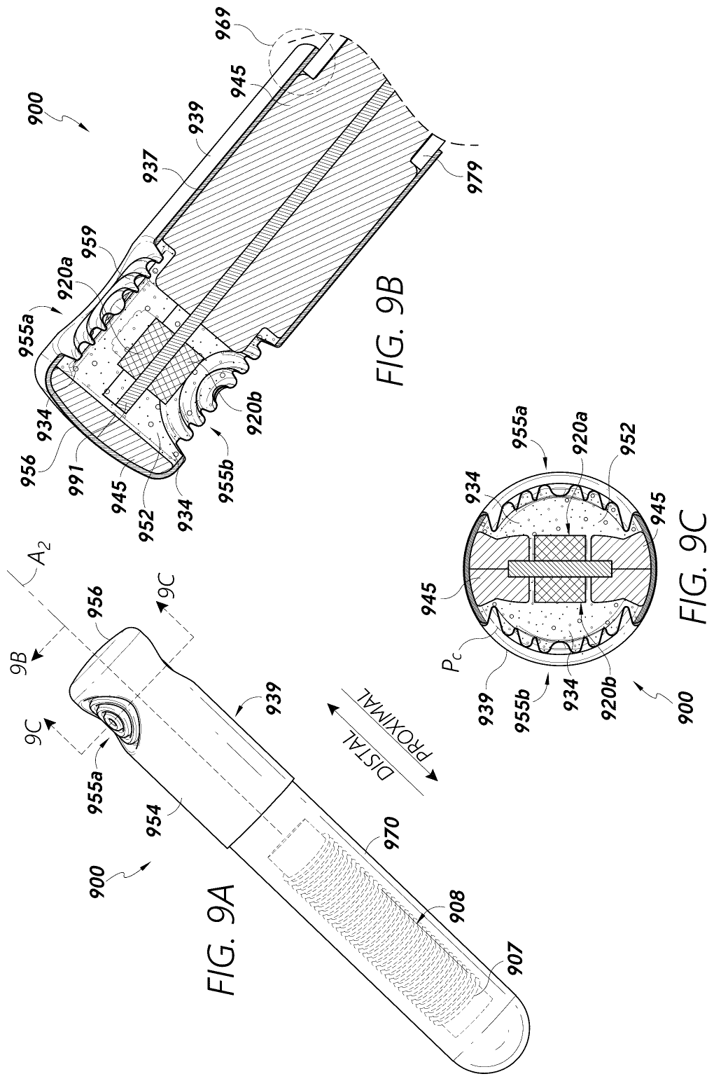

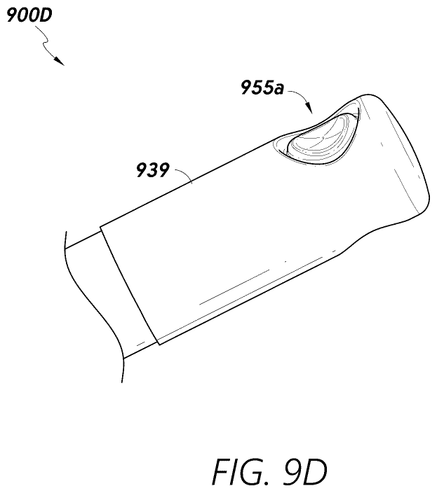

The implant device 30 can comprise certain anchoring structure 31, as referenced above. For example, the anchor structure 31 can include a percutaneously deliverable shunt device configured to be secured to and/or in a tissue wall. Although certain components are illustrated in FIG. 2 as part of the implant device 30, it should be understood that the sensor implant device 30 may only comprise a subset of the illustrated components/modules and can comprise additional components/modules not illustrated. The implant device 30 may represent an example of the implant device shown in FIGS. 9A-9D, and vice versa.

In certain examples, the sensor transducer(s) 32 can be configured to generate electrical signals that can be wirelessly transmitted to a device outside the patient's body, such as the illustrated local external monitor system 42. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some examples.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions and/or provide wireless power using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain examples, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain examples, the local monitor 42 comprises a smartphone, laptop computer, or other mobile computing device, or any other type of computing device.

In certain examples, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or another external subsystem. In certain examples, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain examples, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. Such interrogation can be executed/performed intermittently/sporadically, quasi-continuously, and/or continuously. In certain examples, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some examples, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain examples, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. The local monitor 47, monitoring performed thereby, and/or monitored data can be used/leveraged for troubleshooting. In an example, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain examples disclosed herein describe communication with the remote monitor subsystem 46 from the implant device indirectly through the local monitor device 42, in certain examples, the implant device 30 can comprise a transmitter capable of communicating over the network 49 with the remote monitor subsystem 46 without the necessity of relaying information through the local monitor device 42.

In certain examples, the antenna 48 of the external monitor system 42 comprises an external coil antenna that is matched and/or tuned to be inductively paired with the antenna 38 of the internal implant 30. In some examples, the implant device 30 is configured to receive wireless ultrasound power charging and/or data communication between from the external monitor system 42. As referenced above, the local external monitor 42 can comprise a wand or other hand-held reader. In some examples, the antenna 48 comprises a piezoelectric crystal.

### Implantable Pressure Sensor Devices

Pressure sensors that can be used in medical implant applications include sensors utilizing micro-electromechanical system (MEMS) technology. Such devices may combine relatively small mechanical and electrical components on a substrate, such as silicone or other semiconductor substrate, and may incorporate deformable membranes that are used to measure pressure-induced deflection thereof, wherein the degree of deflection of the membrane is indicative of pressure conditions to which the sensor membrane is exposed at the implant location.

MEMS sensors may be desirable for cardiac implant applications due to their relatively small form factors and packaging. For example, MEMS pressure sensor devices may be considered relatively small, stable, and cost-effective devices, wherein such characteristics can accommodate the relatively constrained space and/or cost requirements of certain implant devices. MEMS pressure sensor devices in accordance with examples of the present disclosure can be fabricated in silicon using certain doping and/or etching processes. Such processes may be performed at a chip-scale, providing relatively small devices that can be co-packaged with certain signal-conditioning electronics, including passive and/or active devices. For example, electronic circuitry electrically coupled to a MEMS pressure sensor in connection with any of the examples disclosed herein may comprise signal amplification, analog-to-digital conversion, filtering, and/or other signal processing functionality and control circuitry.

Various types of pressure sensors can be built using MEMS technology, including piezoresistive pressure sensors and capacitive pressure sensors. Such sensors generally include an at least partially flexible layer that serves as a deformable membrane that is configured to act as a diaphragm that deflects under pressure. Piezoresistive and capacitive sensors use different mechanisms to measure the displacement of such diaphragm components.

With respect to piezoresistive MEMS pressure sensors, certain conductive sensing elements may be fabricated directly onto a diaphragm of the device, wherein changes in the electrical resistance of such conductor(s) can be determined to indicate a measure of pressure applied to the diaphragm. Generally, the change in resistance may be proportional to the strain on the conductor(s), wherein the change in resistance of the conductor(s) is related to the change in length of the conductor(s) induced by deflection of the diaphragm on which the conductor(s) are disposed.

FIG. 3A is a side view of a piezoresistive pressure sensor device 320 in accordance with one or more examples. FIG. 3B is a side view of the piezoresistive pressure sensor 320 of FIG. 3A, wherein a diaphragm 325 of the sensor is deflected in accordance with one or more examples. The deflection of the diaphragm 325 may be caused by pressure conditions to which the diaphragm 325 is exposed. The diaphragm 325 may be formed from a substrate material 326, such as silicon or other semiconductor or material. For example, a trench or cavity 329 may be etched or formed in the substrate 326 to produce a relatively thin membrane for the diaphragm 325.

The diaphragm 325 may have one or more conductive traces or elements 322 disposed thereon and/or applied thereto. For example, the conductive elements 322 may comprise traces of metal or other electrical conductor, wherein one or more length portions of the conductor(s) extend over the diaphragm 325, such that deflection of the diaphragm 325 causes one or more portions of the conductor(s) 322 to elongate/lengthen, thereby altering the electrical resistance/impedance thereof. When the diaphragm 325 deflects, as shown in FIG. 3B, electrical current and/or voltage through the conductive element(s) 322 may be measured to determine respective resistances/impedances thereof, thereby providing a measurement indicating a degree of deflection of the diaphragm 325; such deflection indicates the environmental pressure experienced by the diaphragm 325. The diaphragm 325 may comprise any material(s), including but not limited to metal, ceramic, silicon, and the like.

FIG. 4A is a side view of a capacitive pressure sensor device in accordance with one or more examples. FIG. 4B is a side view of the capacitive pressure sensor of FIG. 4A, wherein a diaphragm of the sensor is deflected in accordance with one or more examples. For capacitive MEMS pressure sensors, one or more conductive layers 421, 422 may be deposited/applied on/to the diaphragm 425 and at the bottom of a cavity 429 behind/below the diaphragm 425, respectively, to create a capacitor. For example, in some implementations, the diaphragm itself 425 may comprise conductive material serving as a capacitor electrode, or a separate conductive electrode may be applied to a side of the diaphragm 425 that is exposed within the cavity 429. That is, the sensor device 420 may comprise one rigid plate electrode 422 and one flexible membrane electrode 425. With the area of such electrodes being fixed, the capacitance between the electrodes may be proportional to the distance(s) between them.

As shown in FIG. 4B, inward/downward deflection/deformation of the diaphragm 425 may change the spacing between the conductors 421, 422 over at least a portion of the diaphragm 425, thereby changing the capacitance of the capacitor formed between the diaphragm 425 and the base electrode 422. Such change in capacitance may be measured by coupling the sensor device 420 to a tuned circuit, for example, which may have a fundamental frequency that is proportional to the degree of deflection of the diaphragm 425. The diaphragm 425 may comprise any material(s), including but not limited to metal, ceramic, silicon, and the like.

### Implantable Pressure Sensor Packaging

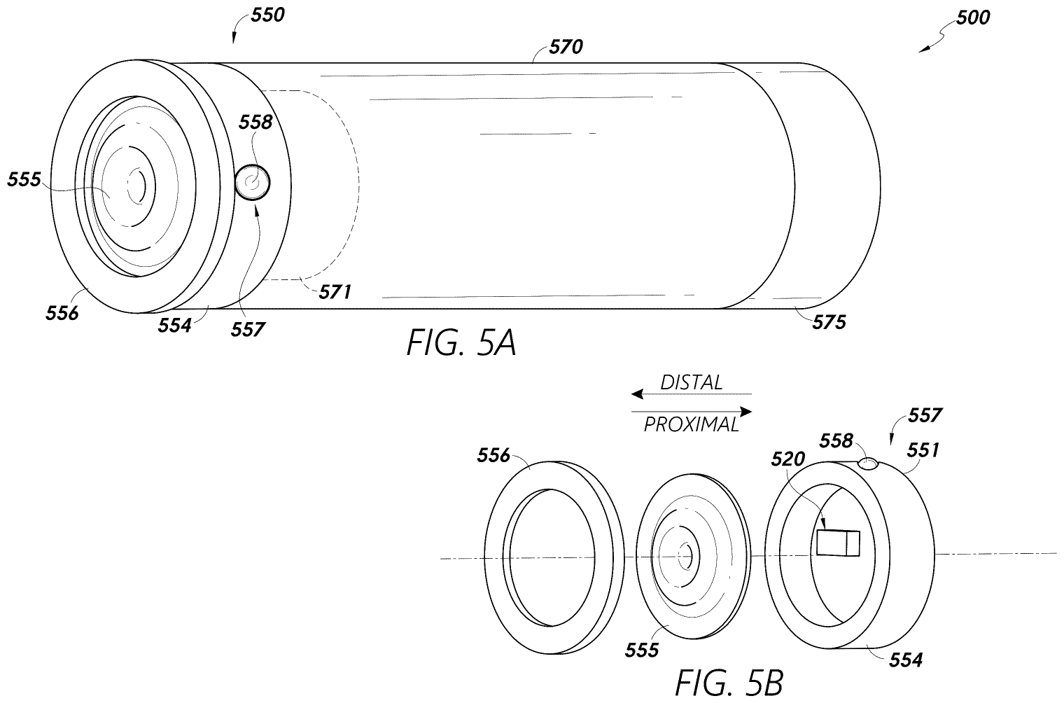

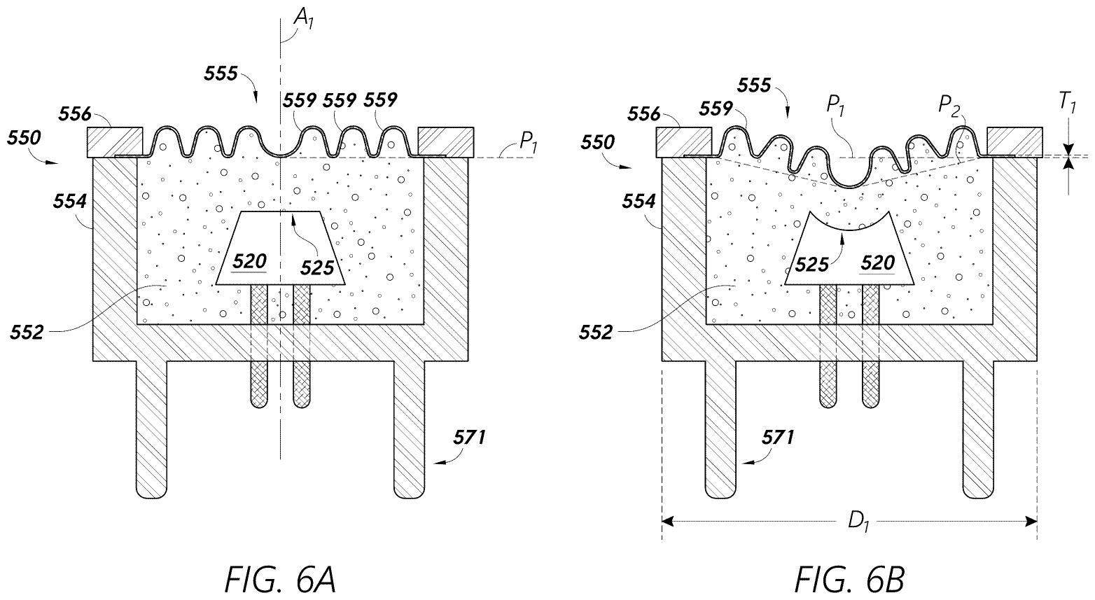

FIG. 5A shows a front and side perspective view of a sensor implant device 550 in accordance with one or more examples. FIG. 5B provides an exploded view of the sensor can package of the sensor implant device 550 of FIG. 5A in accordance with one or more examples. FIGS. 6A and 6B show cross-sectional views of the sensor implant device 550 in accordance with one or more examples. The description below of the sensor device 550 can be understood with reference to any of FIGS. 5A, 5B. 6A, and/or 6B.

The sensor device 550 can be utilized for certain pressure monitoring applications and can utilize relatively low-cost MEMS sensor(s) 520, which are protected from the environment with pressure-transmission fluid/medium 552 in contact with a secondary diaphragm/membrane 555. Such secondary diaphragm 555 may be made from a material that is suitable for placement in relatively corrosive environments with repeated cycling, as may be the conditions in certain vessels/chambers associated with the cardiac system. For example, the sensor can 550 may be suitable for implantation within a patient's cardiac system (e.g., chamber of the heart). The secondary membrane 555 and can structure 554 of the device 550 may comprise metal components, which may be welded, brazed, cold-formed, bonded, or otherwise coupled together in a manner as to create a fluid-sealed pressure-transmission cell, as shown.

The sensor element 520, which may comprise, for example, a MEMS pressure sensor, may be disposed within the can/cell 554, wherein the transmission fluid or other medium (e.g., oil, gel, epoxy) is disposed about the sensor element 520 within the can structure 554, such that the pressure-transmission medium 552 is sealed within the can structure 554 and disposed about the sensor element 520 within the outer enclosure. A portion of the enclosure 550, such as a distal axial area thereof with respect to the particular example illustrated in FIGS. 6A and 6B, comprises the diaphragm component 555.

In terms of manufacturing of the sensor device 550, mechanical welding/coupling may be necessary around the diaphragm 555, as well as around the base 571 of the can 554. For example, with reference to FIG. 5A, the can base 571 may be welded/coupled to the housing 570. Such welding/coupling may introduce complexity into the manufacturing process and/or potential opportunity for defective sealing. As sensor device designs are reduced in size to allow greater flexibility with respect to minimally-invasive (e.g., transcatheter) delivery and implantation within the body, the materials and processes associated with such devices can require increased processing complexity, failure modes, and/or place ultimate limits on further reduction. The housing 570 may be at least partially transparent to electromagnetic radiation.

The diaphragm 555 may advantageously be deflectable, such that pressure conditions external to the enclosure 554 can cause inward deflection of the diaphragm 555 in a manner as to exert pressure on the sensor element surface/diaphragm 525. For example, the pressure-transmission medium 552 may comprise an incompressible fluid or medium in some examples. Alternatively, the medium 552 may be compressible, wherein deflection of the diaphragm 555 may cause a reduction in volume of the internal chamber of the can 554, thereby compressing the fluid/medium and resulting in increased pressure within the can 554 that is translated to the sensor element 520. The deflection of the diaphragm 555 may cause the diaphragm 555 to move from a non-deflected state or configuration in which the diaphragm lies in or primarily parallel to a transverse plane P; (e.g., transverse with respect to an axis of the diaphragm and/or sensor device) to a deflected state or configuration (see FIG. 6B) in which the diaphragm 555 conforms to a concave/deflected plane P₂ that is deflected relative to the transverse plane P₁ in a direction, e.g., toward the sensor element 520.

In order to allow for in vivo implantation, the diaphragm 555 may comprise metal or other material suitable for the relatively harsh environment of the target anatomy. For example, metals (e.g., stainless steel, nickel titanium (i.e., nitinol)), polymers, or other biocompatible materials may be implemented for the diaphragm 555. Furthermore, it may be necessary or desirable to design the diaphragm 555 in a manner as to provide sufficiently high sensitivity with respect to deflection propensity over the expected range of pressure conditions associated with the target physiology. In order to provide increased deflection sensitivity, the diaphragm 555 (or any diaphragm disclosed herein) may comprise one or more corrugations 559. For example, such corrugations 559 may comprise ring-shaped ridges and/or grooves, which may be concentric with the axis A₁ of the diaphragm 555. Such corrugations 559 may provide a sufficiently large linear range for the diaphragm and improved sensitivity. Corrugations may further provide for relatively greater deflection and/or accurate spring rates for the diaphragm and/or extend the cycle life of the diaphragm by reducing mechanical stresses in one or more areas of the diaphragm, depending on the particular corrugation design. The corrugations 559 may be produced by cold-pressing the diaphragm material into the corrugated shape, or by any other means.

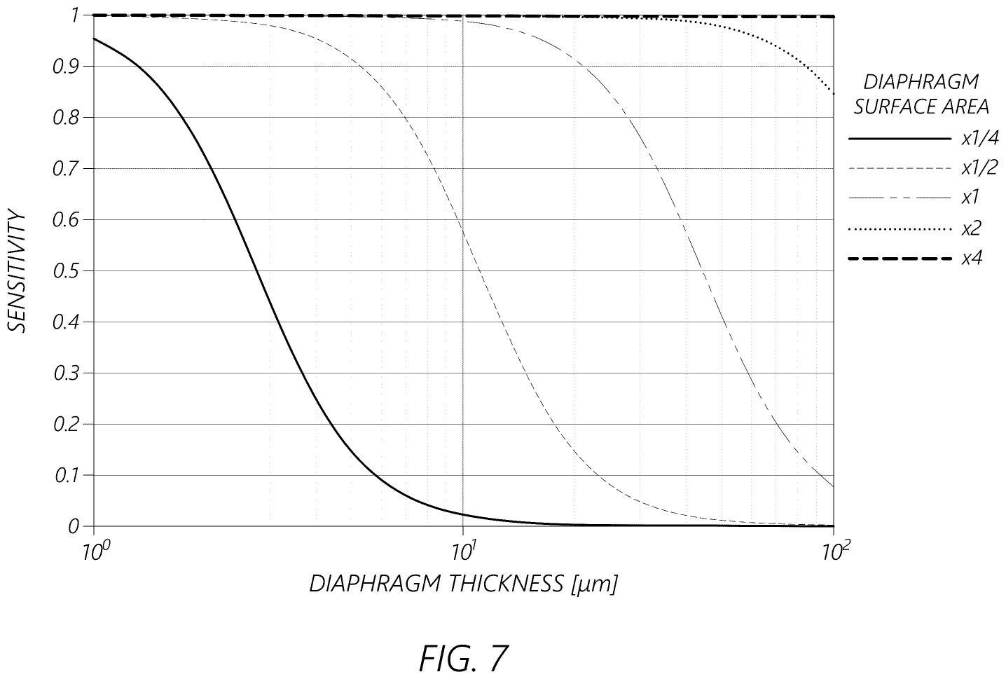

As referenced above, due to size constraints associated with implantable sensor devices, the area available for diaphragm components may likewise be constrained, depending on the design of the sensor device. For example, with respect to axial diaphragms, as in the examples of FIGS. 5A, 5B, 6A, and 6B, the diameter and area of such diaphragms may be constrained by the diametrical dimension D₁ of the device. For example, elongated sensor devices suitable for transcatheter transportation may generally be limited in profile/dimension with respect to the diameter/width thereof, whereas the length of the sensor device may be less constrained in some instances. Therefore, as sensor device profile is reduced, axial diaphragm area may likewise be reduced. As a diaphragm effective areas reduce, it may be necessary to reduce the thickness T₁ of the diaphragm as well in order to maintain sufficient sensitivity in the diaphragm. For example, FIG. 7 is a graph showing relationships between sensor diaphragm thickness, surface area, and sensitivity in accordance with one or more examples. As demonstrated in the graph of FIG. 7, diaphragms having relatively smaller surface area generally must be relatively thinner in order to achieve comparable sensitivity compared to diaphragms having relatively greater surface area and otherwise similar design.

Processes implemented to form thin foils can cause variable strain hardening of the formed materials, which may result in relatively large variation in mechanical performance of a formed diaphragm. Furthermore, relatively thin materials utilized for diaphragm formation can require careful handling and assembly processes to weld the diaphragm structure to the larger sealed body. At such scale, the processes implemented can influence the material properties and mechanics of the diaphragm, thereby causing additional variation in diaphragm performance.

In addition, certain sensor devices are designed with sensor diaphragms positioned/disposed at a distal end of the sensor device assembly, as shown in the examples of FIGS. 5A-6B. Since it can be advantageous to increase the area of the diaphragm to provide desirable sensitivity, as indicated in FIG. 7, increase in diaphragm area for such designs can be at the cost of increasing sensor device diameter/profile, potentially interfering with the ability to fit within a tubular catheter/shaft for delivery, particularly in consideration of diaphragm material thicknesses and deflection sensitivity according to the relationships demonstrated in the graph of FIG. 7. As sensor device designs evolve toward smaller and smaller-profile devices (e.g., millimeter-scale integrated implant devices), the ability to form and integrate such sensor device assemblies can become untenable with respect to the design paradigm demonstrated in FIGS. 5A-6B.

Examples of the present disclosure provide alternative sensor device design solutions allowing for increased diaphragm area while maintaining relatively small-diameter profiles. In some examples, increased sensor diaphragm area is achieved at least in part by positioning sensor diaphragms in a transverse orientation on circumferential wall(s) of a cylindrical sensor body/shell rather than on an axial end of the sensor device. In some examples, diaphragms are included on both cylindrical side walls of the housing as well as on an axial end of sensor device housing/structure. In such cases, the diameter of the diaphragm(s) on the cylinder side wall(s) may be greater with respect to at least one dimension thereof than the diameter of an axially-facing diaphragm of the same device.

Examples of the present disclosure advantageously provide solutions for implantable pressure sensor devices that are relatively small in size, while providing sufficient and/or improved sensor performance/sensitivity by incorporating sensor diaphragms that have sufficiently large area and/or material thickness characteristics to provide such sensitivity. Such examples further provide, in some cases, suitable and/or improved biocompatibility characteristics and/or relatively simplified manufacturing processes. Inventive sensor implant device solutions presented herein can achieve these benefits at least in part through integration of pressure sensor diaphragm components with other structural/mechanical housing/encapsulation component(s) of the device in one or more uniform and integrated layers of deposited material. For example, while certain pressure sensor devices require manufacturing processes that involve multi-part and/or multiple-process manufacturing to seal/mechanically-couple diaphragm components to other structural components, examples of the present disclosure allow for manufacturing without the need for such scaling/coupling step(s)/process(es). Furthermore, in addition to the advantages associated with manufacturing structural and diaphragm components of a sensor device as a single integrated/net component in relatively fewer manufacturing steps/processes, such devices in accordance with aspects of the present disclosure can provide superior mechanical properties relative to certain non-integrated diaphragm solutions. Furthermore, integration of diaphragm and shell components can greatly reduce component count and process steps required to produce the resulting sensor implant device and/or associated packaging. With fewer components and areas requiring hermetic sealing, more robust protective shells can be produced that present a reduced risk of failure/leakage. In addition, pressure sensor devices including integrated diaphragm components, as described in detail herein, can advantageously integrate and improve the consistency and/or transmission performance of sensor membranes/diaphragms that separates the biological environment from the internal sensor components of the sensor device.

Certain examples of the present disclosure provide alternatives to wrought-metal machining, stamping, grinding, or the like, of sensor diaphragms in order to provide diaphragms with reduced thicknesses and improved sensitivity. Such diaphragms may be advantageously formed using an ionized deposition process, rather than through stamping, welding, or other more complicated and/or inconsistent/error-prone processes. Furthermore, deposition of diaphragms in an integrated manner with sensor housing/shell structure can provide improved hermetic sealing, while also involving reduced risk of error due to reduced process variations. In some examples, diaphragms deposited/formed in accordance with aspects of the present disclosure comprise nitinol metal alloy rather than titanium, which may be utilized in other sensor designs.

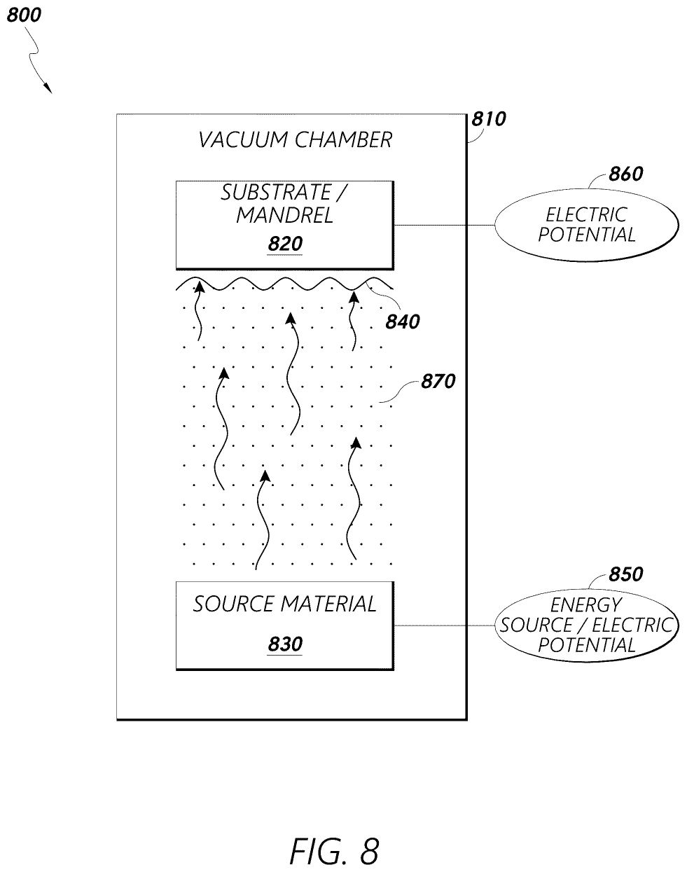

As referenced above, sensor diaphragms in accordance with aspects of the present disclosure may be manufactured/formed using ionized metal vapor deposition in some implementations. FIG. 8 is a block diagram showing a vacuum deposition system 800 in accordance with one or more examples. Physical vapor deposition (PVD) and other vacuum deposition processes can be used to produce relatively thin films and coatings. In the system 800, a source material 830 (e.g., metal) transitions from a condensed phase to a vapor phase 870 and then back to a thin film condensed phase 840. Sputtering or evaporation may be implemented to produce the vaporized/plasma gas 870. The plasma gas 870 is deposited on a substrate 820 to form the layer 840 of the deposited source material. The vacuum chamber 810 may advantageously be devoid of air and particles that could otherwise interfere with the directed deposition onto the substrate 820.

Transformation from solid 830 to gas 870 can be achieved through application of energy from an energy source 850. The energy source 850 may be any type of energy, including heat/thermal current, electrical current, and/or voltage potential relative to the potential 860 associated with the substrate 820. Energy may energize the source material 830 to produce the plasma form 870. The electric potential 860 relative to the source material 830 may serve to create a direction of the deposition flow 870 towards the substrate 820. Source material 830 may be positively charged in some cases, whereas the electric potential 860 of the substrate 820 may be negatively charged.

With respect to the various processes and devices disclosed herein, any type of deposition process may be implemented to produce the inventive shell housing components having integrated diaphragm layer(s). Examples may include, cathodic arc deposition, in which a high-power electric are is discharged at the target (source) material to blast away some into highly ionized vapor to be deposited onto the workpiece. For electron-beam physical vapor deposition implementations, the material to be deposited is heated to a relatively high vapor pressure by electron bombardment in a vacuum and is transported by diffusion to be deposited by condensation onto a relatively cooler workpiece. For evaporative deposition, the material to be deposited may be heated to a relatively high vapor pressure by electrical resistance heating in a vacuum. As another example, close-space sublimation can involve placing the source material and substrate in relatively close proximity to one another and radiatively heated. Pulsed laser deposition may be implemented by ablating the source material into a vapor using a high-power laser. Pulsed electron deposition may be implemented by ablating the source material to generate a plasma under nonequilibrium conditions using a highly energetic pulsed electron beam.

In some example examples, sputter deposition may be implemented, wherein a glow plasma discharge, which may be localized around the target substrate by a magnet, bombards the source material, thereby sputtering some away as a vapor for subsequent deposition. For sputtering applications, a magnetron may be employed that utilize strong electric and magnetic fields to confine charged plasma particles close to the surface of the sputter target. Generally, in a magnetic field, electrons follow helical paths around magnetic field lines, undergoing more ionizing collisions with gaseous neutrals near the target surface than would otherwise occur. The extra ions of the sputter gas created as a result of these collisions can lead to a higher deposition rate. The plasma can also be sustained at a lower pressure this way. The sputtered atoms are neutrally charged and so are unaffected by the magnetic trap. Other sputtering techniques that can be implemented include ion-beam sputtering, reactive sputtering, ion-assisted deposition, high-power impulse magnetron sputtering, gas flow sputtering, or the like.

The substrate/mandrel 820 may be configured to rotate to allow for formation of a three-dimensional form/shell. The rotation may be controlled in a manner as to produce a relatively even distribution of deposition material over the mandrel 820 around the three-dimensional form.

Sensor implant devices having integrated and/or transverse-facing diaphragms in accordance with aspect of the present disclosure may include a housing comprising a shell formed of one or more layers of vapor deposition. For example, such layered deposition can involve depositing a thin layer of deposition material, followed by masking a portion of the deposition (e.g., a diaphragm portion) and subsequently depositing another, possibly thicker, layer. Alternatively, a thicker layer may be deposited over a masked diaphragm (or the diaphragm can subsequently be removed), after which, with no mask over the diaphragm, a relatively thinner layer may be applied to produce the integrated diaphragm and shell structure.

FIGS. 9A, 9B, and 9C show perspective, longitudinal cross-sectional, and axial cross-sectional views, respectively, of a sensor device 900 having an integrated diaphragm(s) 955 in accordance with one or more examples. FIG. 9D shows a view of an alternative sensor device example having a dimple-type diaphragm form 955. The diaphragm(s) 955 and at least a portion of the shell encasement 939 may be formed using a vapor deposition process in one or more stages.

The sensor implant device 900 includes a wireless telemetry component 908. The wireless telemetry functionality associated with implant devices disclosed herein can be configured to transmit and/or receive radiofrequency electromagnetic signals, ultrasound signals, and/or other wireless signal type. Although wireless data and/or energy transmission is described in connection with various examples disclosed herein, it should be understood that such examples may be implemented using wired data and/or power transmission features. For example, the sensor implant devices disclosed herein may be implemented as components of a catheter assembly, wherein such devices may not be intended for long-term implantation, but rather may be positioned in target anatomy through advancements and positioning of such catheter and/or distal end thereof.

The implant device 900 includes a shell encasement 939, which may have one or more MEMS (or other-type) pressure sensor devices 920 mounted or secured therein. The implant device 900 may include a circuit board or other substrate 991 (e.g., printed circuit board) having certain electronics mounted thereto. As illustrated in various figures (see, e.g., FIG. 9A, image 1110 of FIG. 11C), the shell 939 may have a silo-type shape. The silo-type shape may include a first closed (e.g., conical or domed-shape) end/endcap and cylindrical sidewalls and a second end which may be closed open.

In some examples, a wire coil antenna 908 or other type of transmitter/receiver is electrically coupled to the board 991 and/or electrical components mounted thereto. In the illustrated example, a conductive wire coil (e.g., copper wire) may be wound around a ferrite core 907 that is collinear or coplanar with the axis A₂ of the cylindrical sensor housing 970 in which it is at least partially disposed. The ferrite core 907 may be configured to concentrate magnetic field flux, such that the signal radiated by the antenna 908 is concentrated and/or directed. In some examples, the ferrite core 907 can be configured to suppress relatively high-frequency electronic noise. The ferrite core 907 may comprise iron, ceramic, or the like, and may employ relatively high-frequency current dissipation to prevent electromagnetic interference in one or more dimensions.

The various control circuitry components, including the board 991 and antenna 908, may be maintained at least partially within a rigid base housing 970. The tubular base housing 970 may comprise ceramic, zirconia, glass, or other at least partially rigid structure that is hard enough to protect the internal components from damage and fluid and ion ingress during implantation and/or over prolonged exposure at the implantation site. The housing 970 may advantageously provide a complete hermetic seal and moisture barrier to prevent moisture from penetrating the housing 970 and interacting with the components housed therein. The electronics housing 970 may further comprise material that is sufficiently transparent to radiofrequency electromagnetic radiation, which may be transmitted to and/or from the antenna 908 to allow for data and/or power communication with the implant device 900. In some examples, the sensor implant device 900 is configured to communicate data and/or power/energy through transmission of ultrasound signals and/or other sonic signal communication. Therefore, it may be desirable in some examples to construct the housing 970 from material that is sufficiently transparent to ultrasound and/or other sonic signals. In some examples, a distal end 979 of the base housing/tube 970 may be covered with a metal or other sealing shell that seals the distal end of the sensor device 900 and encases the sensor element(s) 920.

In some examples, the shell encasement/housing 939 includes one or more sidewalls/sidewall-portions 954 and a cover/endcap portion 956. The diaphragm(s) 955 may be formed of one or more layers of deposition material that one with one or more deposition layers that form the sidewall(s) 954, such that the interface between the diaphragm(s) 955 and the sidewall(s) 954 is fluid-tight configuration. The shell 939 is secured to the base housing 970 in some manner. For example, a brazing process may be implemented to mount the shell 939 to the base housing 970, which may comprise dissimilar material. Deflection of the diaphragm(s) 955 may be translated to diaphragm(s)/membrane(s) of one or more sensor (e.g., MEMS) devices 920 disposed within the shell package 939 through a transduction medium 934, such as silicone oil.

The diaphragm(s) 955 may have a corrugated form, as shown, or may have any other form. The corrugated topology of the diaphragm 955 may facilitate deflection of the diaphragm 955 in a manner as to translate pressure to the sensor device(s) 920. The shell 939 can be sealed to the base housing 970 to enclose a cavity or chamber within the shell 939. In some examples, the chamber 952 is filled with a liquid material, such as silicone oil or the like, wherein such liquid can be compressible or non-compressible; inward deflection of the membrane(s) 955 increases pressure within the chamber 952 and/or pushes the medium 934 against the diaphragm(s)/membrane(s) of the sensor(s) 920.

In examples in which the chamber 934 is filled with liquid, it may be desirable to fill the chamber 934 such that no air or gas bubbles exist within the chamber. For example, air/gas may generally be pressure-compressible, such that the presence of air/gas within the chamber 952 may decrease the translation of pressure from deflection of the diaphragm(s) 955 into pressure within the chamber 952. In some implementations, the chamber 952 may be filled through a port/channel formed in the internal support structure 945 of the sensor device 900. For example, the port/channel may be used to pipe the liquid into the chamber 952, wherein the port/channel is subsequently sealed in some manner to prevent leakage of the fluid out of the chamber 952 and/or prevent gas or other matter from entering the chamber 952 after it has been filled. In some implementations, the chamber 952 may be filled under vacuum conditions. In some examples, the pressure transduction medium 934 comprises polymer potting, which may be injected or poured/flowed into the shell 939.

In some examples, the board 991 may be held in place by the internal structure 945 of the sensor device 900. A portion of the circuit board 991 may be configured and/or positioned to extend distally passed the end 979 of the housing 970, such that the sensor device(s) 920, which may be disposed on the portion of the board 991 that extends past the end of the housing 979 and into the internal area of the shell 939, is not covered axially by the housing 970, but rather by the shell 939.

The sensor device 900 may have any suitable or desirable diaphragm topology. For example, while the sensor device 900 is shown in FIGS. 9A-9C as having a corrugated diaphragm, as described in detail above, FIG. 9D shows an alt alternative diaphragm design for a sensor device 900D that has a dimpled diaphragm, which may comprise a concave depression defined by a concentric ridge/groove.

The diaphragm(s) 955 may comprise a thin layer of deposited metal, such as nitinol, titanium, or other metal. The relatively thin layer of diaphragm 955 may be a unitary form with at least a portion of the body/sidewall portion(s) 954 of the sensor enclosure shell 939. By integrating the diaphragm 955 with the shell 939, the diaphragm 955 can have an inherent seal with the shell structure encapsulating the sensor(s) 920, thereby providing increased protection for the internal components and/or protection from biological breakdown over time. The diaphragm(s) 955 provide the transmission window through which external pressure conditions are transferred to the internal sensor device(s) 920 via the encapsulated pressure transmission medium 934 (e.g., oil). The diaphragm(s) 955 may be formed of a final deposition layer the provides an exterior skin over the shell 939, which provides structure for holding internal components within the sensor device 900.

Where the exterior layer(s) of the encapsulation shell 939 is formed of deposited metal deposited in unitary form, such deposition can provide structural continuity between the diaphragm(s) 955 and the body 954 of the shell 939. Such continuity can be advantageous for various reasons. In examples in which separate metal components/layers form the diaphragm and the structural housing, slight differences in the metal structure and/or interfaces therebetween can introduce imperfections and/or structural defects in the overall structure of the housing. Therefore forming such components of a common source material and through a common deposition application can provide structural benefits for the shell 939.

The structural shell 939 may be formed of one or more coats, layers, or processes, such that the body portion 954 may have a different thickness than certain other portions, such as the diaphragm 955. For example, in some implementations, an initial relatively thicker layer 937 (e.g. approximately 100-300 μm; 250 μm) may be deposited on a substrate to form the relatively thicker and/or more rigid body 954 of the shell 939 outside of the diaphragm area 955. For example, such layer(s) may be deposited/formed while the diaphragm area 955 is masked. Alternatively, the thicker coating/deposition of metal (e.g., nitinol) may be deposited over the entire shell 939, including the diaphragm portion(s) 955, wherein a thickness of the deposited material on the diaphragm(s) may be etched away or otherwise thinned to produce a relatively thinner diaphragm, which may be desirable with respect to sensitivity/flexibility of the diaphragm. In some examples, the diaphragm thickness may be approximately 10 μm, such that the diaphragm is approximately 3-15% (e.g., 4%, 5%, 10%, or any number therebetween) as thick as the structure of the shell 939 outside of the diaphragm areas (i.e., in the body portion 954 of the shell 939). For example, where the diaphragm 955 is masked during deposition of the thicker layer 937 of the shell 939, after such deposition, the mask may be removed to allow for deposition of the same metal/material over the entirety of the shell 939, including over the portions of the diaphragm(s) and area immediately adjacent thereto, wherein such subsequent layering/deposition may be relatively thin skin/layer (e.g., approximately 10 μm thick, or between 10-40 μm thick).

In some implementations, the thin-layer diaphragm may be deposited over the mandrel/substrate surface, after which, the diaphragm 955 may be masked to allow for subsequent deposition of the thicker layer 937 over the areas of the mandrel/substrate and thin deposition layer outside of the diaphragm area 955 to fully form the shell 939. Removal of the diaphragm mask and the mandrel/substrate to produce the complete integrated shell and diaphragm.

With further reference to the manufacturing process for producing the sensor device 900, as referenced above, in some implementations, a deposition process may be implemented to deposit a thickness/layer of metal or other material (e.g., nitinol) over a mandrel or other substrate, wherein a laser process may be implemented to selectively remove at least a portion of such deposition in the area of the diaphragm(s) 955. For example, in some limitations, substantially all of the deposited material in the area of the diaphragm(s) may be removed. After such diaphragm removal, the structure may be placed back into the deposition chamber (e.g., PVD vacuum chamber), wherein a final skin/layer may be applied over the previously deposited material that has not been removed, as well as over the diaphragm area where the previous deposition was removed/thinned or blocked.

In some implementations, masking of the diaphragm may be implemented by using a resist layer over the diaphragm(s) to prevent buildup of the deposited material in the covered area. By depositing a final integrated layer/coating over the structure including the diaphragms, a robust hermetic seal may be produced along the entire shell 939. That is, the process of applying first a thick film layer over the body of the shell followed by a final thin film coating over the diaphragms and the body of the shell, including the area surrounding the diaphragm(s), can provide desirable adhesion between the thin outer skin/diaphragm layer and the remaining structure, provided that the surface of the structure is maintained relatively clean between process stages and the same deposition material is applied in both stages. In instances where contamination is permitted between process steps, adhesion may be compromised, although in some instances the structural integrity may be sufficient to meet the particular needs. The deposition material used to form the layer(s) of the shell 939 include nitinol, titanium, stainless steel, and other metals having similar properties. The base housing component 970, which is coupled to the shell 939 to form the enclosed housing container, can include zirconium, ceramic, or other material having similar properties. The base housing 970 can be bonded or brazed to the shell 939 in a manner as to maintain a hermetic seal between the two components of the device 900.

The shell 939 formed of the various deposition process steps/stages may be removed from the mandrel and/or other substrate material to produce the hollow shell 939 that is configured to enclose/contain internal sensor elements/components. The shell 939 may then be slid/placed over or within the open end of the open cylindrical base housing 970, which may be configured to hold various internal components of the sensor device 900. For example, while the shell 939 may be comprised of electrically conductive material (e.g., metal, such as nitinol), it may be desirable for the housing 970 to not comprise electrically conductive material. For example, in implementations in which the device 900 is configured for wireless data and/or power transmission, one or more antenna/transmitter elements 908 may be disposed within the base housing 970. Therefore, the base housing 970 may advantageously comprise material that is transparent to wireless electromagnetic signal transmission, such as glass, ceramic, polymer, or other material. Therefore, the combined assembly of the device 900 may advantageously provide both metal and non-metal housing components, which may facilitate desirable combination of biocompatibility, mechanical integrity, and wireless transmission transparency. In some examples, the antenna 908 comprises one or more coils wound about a magnetic core 907 (e.g., ferrite core), which may provide desirable mechanics for data and/or power transmission.

The internal components of the device 900 include a board or other substrate 991 on which the sensor device(s) 920 can be mounted. The board 991 may be held in place by certain spacer/structural component(s)/material 945. For example, the spacer material 945 may comprise epoxy, or material is more solid, such as polymer/plastic materials. The spacer material 945 may provide structural integrity for the device 900, and may be utilized to hold, secure, and/or protect certain internal components of the device. Furthermore, the internal spacer/structural component(s) 945 may serve to secure the shell 939 to the housing 970 and/or help to retain the cylindrical form of such components under stresses/pressure. In some examples, the spacer/structural component(s) 945 comprises a slug form configured to be placed/disposed within the housing 970 in a manner as to allow for the placement of the shell 939 over the slug and housing 970.

The illustrated example of the sensor device 900 comprises a dual-diaphragm shell 939, as well as multiple (e.g., two) internal sensor elements 920, with the first sensor 920a disposed behind a first diaphragm 955a and a second sensor device 920b disposed behind a second diaphragm 955b. Both sensor devices 920 may be mounted to the board 991 or other structural component(s).

The diaphragm(s) 955 are in communication with the internal fluid/medium 934, which transmits deflection of the diaphragms into increased pressure and/or force within the fluid-filled chamber(s) 952, thereby producing pressure conditions that are readable by the sensor device(s) 920 that reflect external pressures. In some examples, the shell 939 is sealed in the area 969 that overlaps the housing 970 to provide a hermetic seal between such components. For example, the slug/body component(s) 945 can seal against the inner diameter of the shell 939. Unlike certain other solutions, as described in detail above, only a single area of sealing of the sensor components may be necessary in order to provide a complete hermetic seal of the assembled device 900. Such seal may be, as referenced above, between the shell 939 and the housing 970 in the area 969 where such components overlap. The seal between the shell 939 and the housing 970 may be created in any suitable or desirable manner, such as through the use of curing of certain materials at specified rates, through adhesive application, brazing, or the like.

The diaphragm(s) 955 may have any suitable or desirable shape or form, including corrugated diaphragms, as shown in FIGS. 9A-9C, dimpled/depression diaphragms as shown in the alternative example shown in FIG. 9D, flat diaphragms (e.g., flat along the curvature of the cylindrical form of the shell 939, or diametrically flat across an arc segment of the cylinder body 954. For example, the diaphragm(s) may be formed of a layer of material that is deposited on and lies in a plane that is tangential to the circumference/perimeter of the cylinder, which is sunk radially within the outer diameter of the cylinder.

As described above, the diaphragm(s) 955 are advantageously transverse-facing. That is, the face/plane of the diaphragm(s) 955 may face in a direction that radially outward from the axis A₂ of the sensor device 900 and shell 939. Transverse orientation of sensor diaphragms can allow for implementation of relatively larger-area diaphragms compared to diaphragms limited to the axial end of the shell/sensor. For example, whereas the area of the endcap 956 may be limited by the diameter of the sensor/shell, the area of the sidewall portions 954 of the shell 939 can correspond to an areas that are larger than the area of the endcap 956. Furthermore, the length of the shell 939 can be designed to any desirable length suitable to accommodate a larger-sized diaphragm, wherein such length may be increased without necessitating an increase in the diameter of the shell/sensor. Because a delivery catheter in which the sensor device 900 is transported may be elongated, the increase in the length dimension of the shell 939 may be less disadvantageous than a commensurate increase in the side of the endcap diameter, and therefore, transverse-facing diaphragms can be relatively larger for small-profile sensor devices.

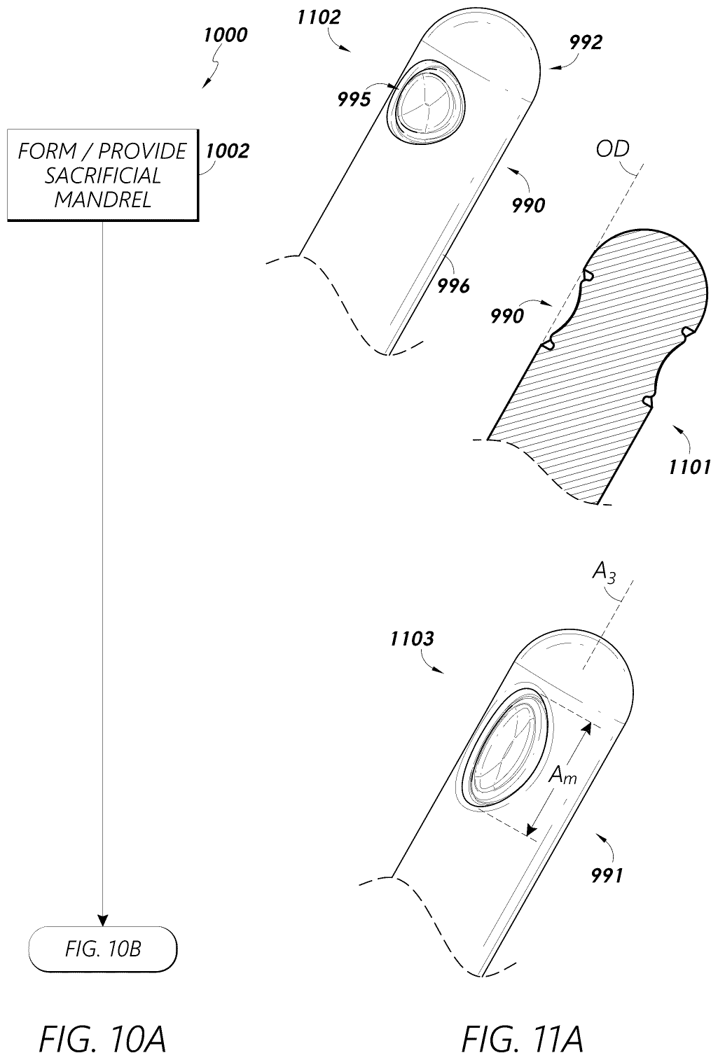

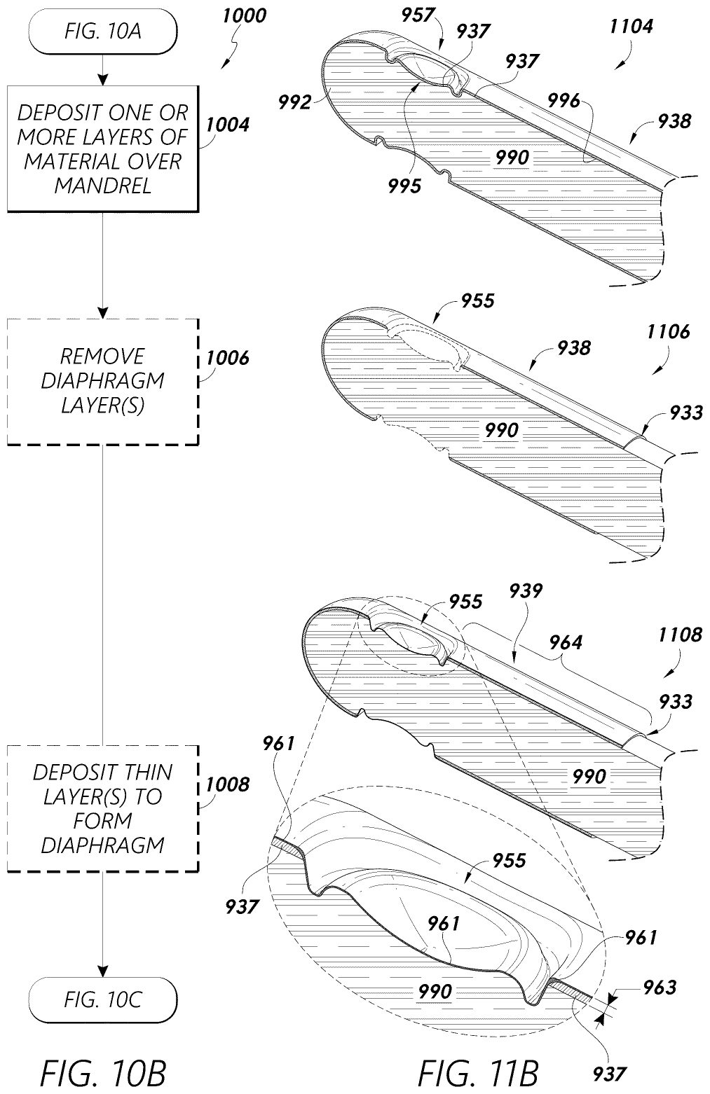

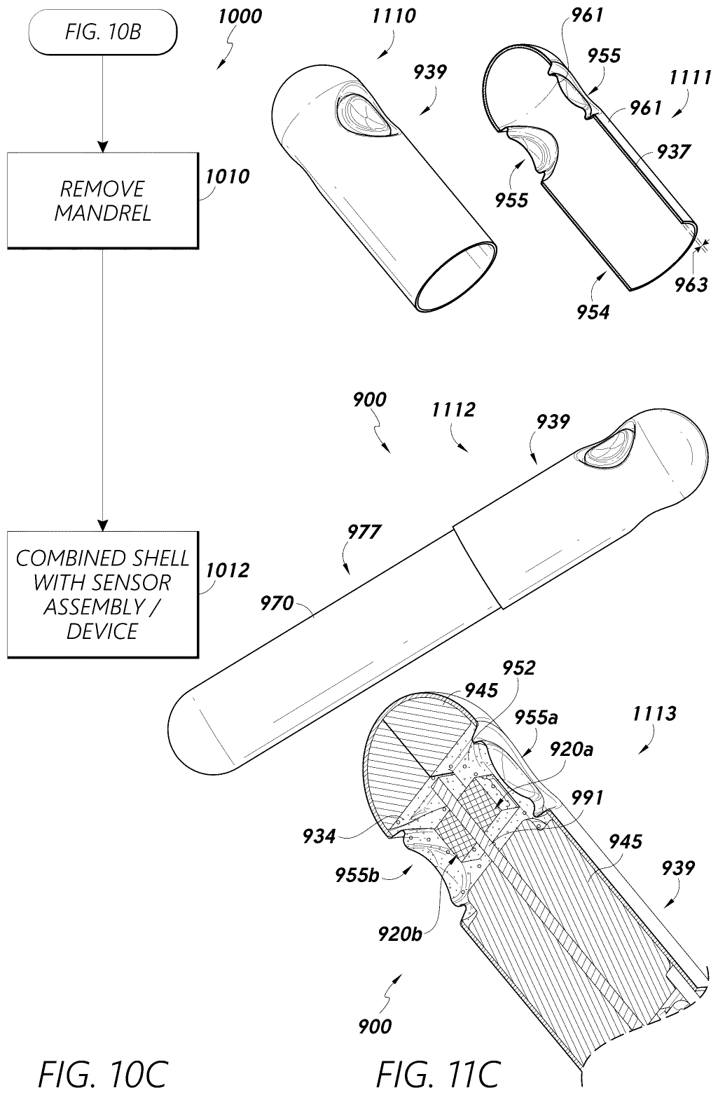

FIGS. 10A-10C provide a flow diagram illustrating a process 1000 for packaging a sensor device in accordance with one or more examples. FIGS. 11A-11C provide images of pressure sensor packaging corresponding to operations of the process of FIGS. 10A-10C according to one or more examples.

At block 1002, the process 1000 involves forming or providing a sacrificial mandrel 990 for formation of a sensor shell as described in detail herein. For example, the sacrificial mandrel 990, shown in image 1102 of FIG. 11A, can comprise material configured to be selectively etched away after deposition on an outer surface thereof of the relevant process material. The mandrel 990 may comprise stainless steel in some examples. The use of an electrically conductive material may be desirable to facilitate direction of deposition vapor in a vacuum chamber, which may be reliant upon implementation of an electric potential between the source material and the mandrel substrate.

The mandrel 990 may be primarily cylindrical in shape, and may be solid or hollow. Examples incorporating hollow mandrels may be advantageous as requiring less sacrificial material etching/removal when deposition is complete. The mandrel 990 may include a dome-shaped and/or flat endcap portion 992. That is, although a semi-sphere dome-type form is shown as the endcap portion 992 in image 1102, it should be understood that the end portion 992 may have any suitable or desirable shape. Furthermore, although the mandrel 990 is shown as having a cylindrical shape, in some implementations, the mandrel (and resulting sensor shell deposition) as a rectangular or elliptical/oval shape.

The mandrel 990 can include diaphragm portion(s) 995 having shape/form corresponding to a desired diaphragm shape or form, such that deposition of material over such areas can produce the desired diaphragm shape/form for the sensor shell when the conformal deposition is applied to the surface thereof. Although examples are disclosed herein in the context of sensor devices including substantially circular diaphragms, it should be understood that any shape of the diaphragm may be implemented, including elliptical diaphragms, as shown in image 1103 of FIG. 11A with respect to the illustrated alternative mandrel 991. In the example of image 1103, the major axis Aₘ of the diaphragm is parallel to the axis A₃ of the shell. As shown from the cross-sectional side view of image 1101 of FIG. 11A, the diaphragm 995, as defined by the form of the mandrel 990, may be stepped-back from the outer diameter OD of the cylinder to provide protection for the diaphragm from the implant environment and/or structural damage associated with manufacturing, delivery, and maintenance in vivo.

Although a three-dimensional mandrel form is illustrated and described, wherein the shell is formed by depositing a three-dimensional cylindrical form over the mandrel, it should be understood that examples of the present disclosure may implement two-dimensional deposition of a sheet of material shaped and configured for post-deposition forming of the three-dimensional shell from the sheet of deposited material. Therefore, any example disclosed herein may be understood to comprise a cylindrical sensor shell deposition or deposition of a sheet that is later formed into a sensor shell. However, forming a shell from a sheet of material can introduce complexity in the manufacturing process and complicate the scaling of the shell for biocompatibility purposes.

At block 1004, the process 1000 involves depositing one or more layers of material over the mandrel 990. For example, such layer(s) may be applied over the exterior surface of the mandrel 990 using physical vapor deposition or other deposition or sputtering process, as described in detail above. The deposition material may be any suitable or desirable material compatible with deposition processes as disclosed herein. For example, the deposited layer(s) of material 937 may comprise nitinol, titanium, and/or other metal.

The operation(s) associated with block 1004 may involve using physical vapor deposition to form a shell of material over the sacrificial mandrel 990 and/or over at least a portion thereof. The target material may be applied to the mandrel 990 in one or more layers to create a relatively even deposition of material 937 over the surface of the mandrel 990, such as over substantially the entire surface thereof with respect to one or more perspectives or angles of the mandrel. In some implementations, the initial deposition of layer(s) of shell material is applied over the diaphragm portion 995 of the mandrel 990 as well as the sidewall 996 and/or end 992 portions. The initial layer(s) 937 of deposition material may be approximately 100-300 μm in thickness such as between approximately 150-250 μm in some implementations. For example, the thickness may be between about 150-180 μm in some implementations.

A particular deposition process implemented may utilize sputtering or evaporation to generate a vapor in the form of molecules or ions from a target source material. Such vapor may be transported and deposited onto the exterior surface of the sacrificial mandrel 990 to create a coating 937. The process of deposition may continue until the desired thickness is achieved. In some implementations, planer magnetron physical vapor deposition is implemented to deposit the layer(s) 937 on the mandrel 990. Such processes can advantageously produce relatively flat application layers. Furthermore, inverted cylindrical magnetron deposition processes can advantageously allow for deposition to occur over a three-dimensional cylindrical surface, as is the mandrel 990 illustrated and described in connection with FIGS. 10 and 11. Deposition layer(s) created by such processes can produce relatively more pristine and isotropic layers compared to wrought forms/materials that are cut, machined, ground, or stamped into the shell form having a diaphragm as described herein.

At block 1006, the process 1000 may involve removing one or more layers of material in the area of the diaphragm(s) 955 to completely remove the diaphragm portion(s) 957 of the shell 939 formed from the initial deposition layer(s) 937, or at least some of the thickness thereof, to produce a relatively thin diaphragm deposition, or completely remove the deposition, over the diaphragm portion(s) 995 of the mandrel 990.

In some implementations, a femto-second laser is utilized to mill-out the diaphragm area 957 of the shell layer(s) 937. Such milling may be performed in any suitable or desirable manner. For example, the diaphragm portion 957 of the shell layer(s) 937 may be completely removed, thereby exposing the diaphragm portion 995 of the mandrel 990 beneath. Alternatively, only a portion of the thickness of the diaphragm portion 957 of the shell layer(s) 937 may be removed in some implementations, or selective areas or thicknesses of the deposited shell layer(s) 937 may be removed, wherein certain areas of the diaphragm portion 957 may be removed down to the substrate 990, while some deposited material is selectively left behind in the diaphragm area 957, thereby allowing/providing a relatively high degree of customization with respect to the mechanics of the final diaphragm component. The material removal associated with block 1006 can advantageously involve removing material that follows the contours/topology of the diaphragm portion 995 of the mandrel 990, whatever shape is implemented therefore. The contours around the diaphragm 995 of the mandrel 990 may advantageously conform to minimum-angle requirements for even deposition according to the implemented deposition process.

Ultimately, the sensor shell 939 formed will include a diaphragm 955 that is thinner than the sidewalls 937 of the shell 939. The deposition material properties can also allow for relatively precise high-resolution laser milling and ablation, potentially allowing for sub-micron tolerances of the removed material.

In some implementations, the produced shell 939 with removed/missing diaphragm portions as shown in image 1106 of FIG. 11B may be produced in any suitable or desirable manner. For example, previously-deposited layer(s) 937 of the shell 939 over the diaphragm 995 may be removed or thinned, as described above. Alternatively, in some implementations, a masking structure or agent may be disposed/applied over the diaphragm region(s) 995 of the substrate 990 when the layer(s) 937 of the shell 939 are initially deposited. The mask may then be removed to expose the diaphragm portion(s) 995 of the mandrel 990 to allow for further deposition in such areas.

In implementations in which laser milling or other process is implemented to produce the desired thickness for the diaphragm 955, the formation of the final shell 939 may be complete following the operation(s) associated with block 1006. In some implementations, laser milling may further be utilized to remove the deposited material in the area of the open edge of the shell 939 to produce a proximal edge 933 that has a relatively clean axial line about the circumference of the mandrel 990.

In implementations in which the mandrel and shell 939 are removed from the vacuum deposition chamber for the purpose of laser etching or otherwise removing material in the area of the diaphragm 957, wherein further deposition layer(s) are intended for deposition on the shell 939, such removal and/or replacement from/in the vacuum chamber may negatively impact adhesion between the initially deposited layer(s) 937 and any subsequently-deposited layer(s). However, such detrimental effects may nevertheless be desirable as a means of achieving the integrated diaphragm structure with a relatively thin diaphragm as described in connection with the examples disclosed herein. The diaphragm area 957 may be selectively etched away in a manner as to avoid etching away portions of the sidewall and/or end portions of the shell 939.

In some implementations, the process 1000 involves, at block 1008, depositing one or more relatively thin layer(s) over the shell 939 and diaphragm portion(s) 995 of the mandrel 990 (e.g., in implementations in which the mandrel 995 is exposed in the diaphragm areas).

The additional secondary layer(s) 961 applied over the shell 939 and diaphragm 995 can be for example, between 10-50 μm thick. Such thickness 961 may be combined with the thickness 937 of the end and sidewall portions to form a relatively thick 963 sidewall and endcap structure for the final shell 939, while producing a relatively thinner diaphragm 955, which may be between 3-20% as thick as the combined layer(s) 963 in the sidewall portions. The new deposited skin/layer(s) 961 can include the relatively thin diaphragm 955 integrated with a thin layer 961 that is laid over the previously-deposited shell 937/939 in the sidewall portions 964, wherein the new/outer layer/skin 961 bonds to the outer surface of the shell 937/939 to create a single/integrated body for the shell 939. The material used for the second deposition 961 associated with block 1008 may advantageously be the same deposition material as applied in the initial deposition process, which may improve adhesion and integration between the layers. The different layers of deposition may be bonded at the molecular level, thereby providing desirable integration. The diaphragm layer(s) may be electro-deposited in some implementations. Furthermore, the surfaces of such deposition may be configured to reduce tissue growth and adhesion thereon in some implementations.

In some implementations, as an initial deposition operation/step, a thin layer is deposited over the mandrel 990, including over the area of the diaphragm 995. After deposition of such thin layer over the diaphragm 995 and surrounding area(s) (e.g., end 992 and/or sidewall 996 portions), the diaphragm area 995/955 may be masked-off, such that the thin diaphragm layer 955 is protected from further deposition/layering. With the diaphragm 955 masked, additional layer(s) of deposition material may then be deposited over the mandrel and/or previously-deposited thin layer of shell. Such additional deposition may be relatively thicker than the thin diaphragm layer, thereby producing the relatively thick shell 937. The masking may then be removed, thereby revealing a relatively thin diaphragm layer 955 integrated with a relatively thicker shell structure 937. The operations described in this paragraph may be implemented in whole or in part as an alternative to, or in addition to, the operations shown in FIG. 10B, or as example implementations of various operations shown in FIG. 10B.

Although some examples are disclosed in which the primary 937 and secondary 961 deposition layer(s) comprise the same material (e.g., metal or metal alloy), in some implementations, the shell 939 may be formed of primary 937 and secondary 961 depositions that comprise different materials, wherein such layers may comprise materials selected to improve or tune the mechanical properties of the shell 939. For example, the different layers may include an outer layer 961 designed to provide suitable biocompatibility properties, corrosion resistance, super elasticity, or other feature(s), whereas the internal layer(s) 937 may be designed to provide desired structural rigidity/strength for the shell 939. The deposited shell 939 provides a unique design with transverse/radial diaphragms.

At block 1010, the process 1000 involves removing the sacrificial mandrel 990. For example, the mandrel substrate (e.g., stainless steel, copper, or the like) may be removed from the completed deposited shell/form 939 to leave the hollow shell. With the internal mandrel etched-out, the complete net form of the shell 939 may be left with a relatively thick structural body 954 and a relatively thinned/thin diaphragm 955. Removing the mandrel can involve dissolving the mandrel form using any suitable or desirable type of etchant.

When the mandrel 990 is etched away, the complete net shell form 939 is isolated, wherein the shell 939 includes a structural body configured for connection to base component 970 of a sensor device housing, as described herein. The shell 939 includes a tuned thin layer 961 in the diaphragm area 955 to provide desirable pressure transduction functionality, wherein the diaphragm 955 is substantially integrated at the molecular level with the body 954 of the shell 939 without welding, brazing, or other bonding method, which, for reasons described above, can introduce undesired complexity and/or risk of error into the process.

The image 1110 in FIG. 11C shows the formed shell 939 with the sacrificial mandrel removed from therein. As apparent in the cross-sectional view of image 1111, the shell 939 is substantially hollow after removal of the mandrel. The cross-sectional image 1111 shows the relatively thicker body portion 954 integrated with the thinner diaphragm portion 955.

At block 1012, the process 1000 involves combining the formed shell 939 with the base sensor component 977 which includes a cylindrical (or other shape) open partial capsule 970 configured to house certain electronics, such as an antenna and/or other circuitry for wireless sensor data and/or power transmission. When combined with the base sensor assembly 977, the sensor device 900 may be fully assembled. In some implementations, a sealing or brazing process may be implemented to secure and/or sealed the shell 939 to the housing 970.

The base assembly 977 may be combined with the shell 939, wherein a plug or other structure 945 is disposed at least partially within the base housing 970, wherein such structural feature is configured to hold or secure certain electronic and/or other components within the sensor device 900 and or otherwise provide structural support/integrity for the assembled device 900. The sensor device 900 shown in images 1112 and 1113 of FIG. 11C may have any of the features and or characteristics of the sensor device shown in FIGS. 9A-9D. For example, the sensor 900 in image 1113 may include a board or other substrate 991 held in place by the structural component(s) 945 and having disposed on one or more sides thereof sensor device(s) 920, such as piezoresistive and/or piezo-capacitive pressure sensor devices, or resistive or capacitive pressure sensor (e.g., MEMS pressure sensor device(s)). Oil or other pressure-transduction means/medium 934 may be injected into the space 952 around the sensor device(s) 920 and between the diaphragms/membranes of the sensor device(s) 920 and the diaphragm(s) 955 of the shell 939 to thereby allow for deflection of the diaphragm 955 to increase pressure against the sensor device(s) 920 to allow for pressure sensing of the external environment. The oil or other medium 934 may be injected/placed in the internal chamber(s) 952 in any suitable or desirable manner. For example, the structure 945 within the capsule 900 may include a fluid injection port providing fluid access to the internal volume 952 around the sensor device(s) 920. The diaphragm(s) 955 may have the illustrated dimple-/speaker-type shape or form, or maybe corrugated or have any other shape or form (e.g., flat/curved).

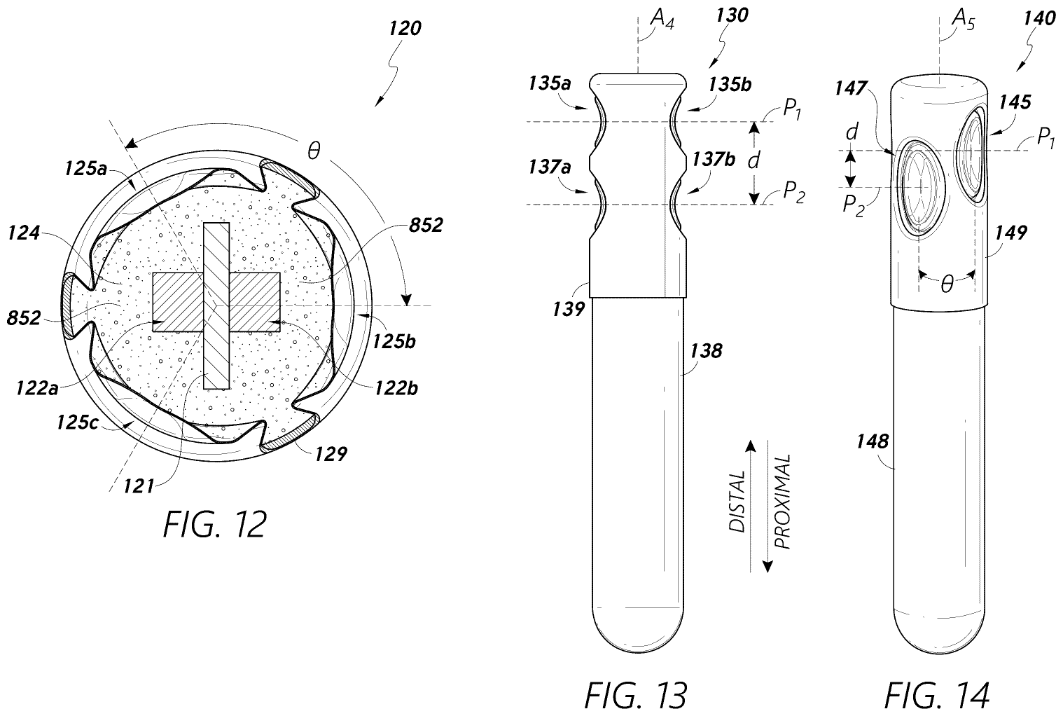

FIG. 12 shows an axial cross-sectional view of a sensor device 820 comprising more than two circumferentially-distributed diaphragms 125 in accordance with one or more examples. As described in detail above, various examples are disclosed herein comprising two circumferentially-distributed diaphragms, wherein the diaphragms are positioned on opposite sides of the perimeter of the cylindrical sensor-encapsulating shell, such that the diaphragms are separated by approximately 180°. It should be understood that examples disclosed herein may comprise more than two circumferentially distributed diaphragms. For example, the sensor device 820 of FIG. 12 includes three circumferentially-distributed diaphragms 125, which are angularly separated by an angle θ. In some examples, the angle of separation θ represents an even division of the radians around the circumference of the shell 129. That is, for a three-diaphragm example, the angular separation θ may be equal to approximately 120°.

Although the illustrated example of FIG. 12 includes three circumferentially-distributed diaphragms, it should be understood that more than three circumferentially-distributed diaphragms may be implemented. In some examples, four circumferentially-distributed diaphragms are implemented in a common axial plane. In such examples, the angle of separation θ between adjacent diaphragms may be approximately 90°, such that the diaphragms are evenly distributed about the perimeter of the cylindrical shell.

Examples comprising more than two circumferentially-distributed diaphragms may include any number of internal sensor devices 122. With respect example of FIG. 12, the internal chamber 852 within the shell 129 may comprise two sensor devices 122a. 122b disposed on opposite sides of a board or other substrate 121. Alternatively, a single sensor device may be contained in some examples. In some examples, the number of sensor devices 122 implemented is equal to the number of diaphragms integrated in the shell 129. For example, with respect to the particular example of FIG. 12, the internal chamber 852 may contain three sensor devices.

FIG. 13 shows a side view of a sensor device 130 comprising a plurality of axially-distributed diaphragms in accordance with one or more examples. As described in detail above, various examples are disclosed herein comprising two or more diaphragms integrated with a sensor-enclosing shell, wherein the diaphragms are positioned in a common axial plane. That is, the diaphragms are axially aligned with one another with respect to an axis A₄ of a cylindrical (or rectangular cuboid) sensor device. In the example of FIG. 13, the sensor device 130 includes a plurality of axially-offset sets of diaphragms, wherein each set of diaphragms is axially aligned with a separate axial plane. For example, the distal-most set of diaphragms 135 may be aligned with one another and centered about a first axial plane P₁, whereas the second set of diaphragms 137 are disposed proximal to the first set 135 and are aligned and centered about a second axial plane P₂ that is axially offset from the first plane P₁ in the proximal direction. Although two diaphragms are shown in each set of axially-offset diaphragms, it should be understood that any number of diaphragms, including one diaphragm, may be implemented in each axial position P₁, P₂. Furthermore, although two sets of axially-offset diaphragms are illustrated, it should be understood that sensor devices in accordance with aspects of the present disclosure may include three or more actually-offset sets of diaphragms. The diaphragms 135, 137 are integrated with a shell 139 which may be formed using deposition process that creates a unitary layer including the side wall portions of the shell 139 as well as the diaphragms.

FIG. 14 shows a perspective view of a sensor device 140 having axially- and/or circumferentially-offset diaphragms in accordance with one or more examples. In the example of FIG. 14, the shell 149 includes multiple diaphragms 145, 147 that are axially offset from one another by a distance d, wherein the first diaphragm 145 is centered about a first axial plane P₁, whereas a second diaphragm 147 is centered about a second axial plane P₂ that is proximal with respect to the more-distal plane P₁. Unlike the example of FIG. 13, the axially-offset diaphragms 145, 147 may be at least partially axially overlapped. Furthermore, the diaphragms 145, 147 may be circular, or oval/oblong in shape, as in the illustrated example. The diaphragms 145, 147 are further illustrated as being circumferentially offset with respect to the axis A₅ of the device 140 by an angular offset θ. The angular offset θ between the diaphragms 145, 147 may be about 180°, such that the diaphragms are on substantially opposite circumferential sides of the shell 149, or maybe offset by an amount less than 180°, such that both diaphragms 145, 147 are disposed at least partially on a common diametrical side of the shell 149, as in the illustrated example of FIG. 14.

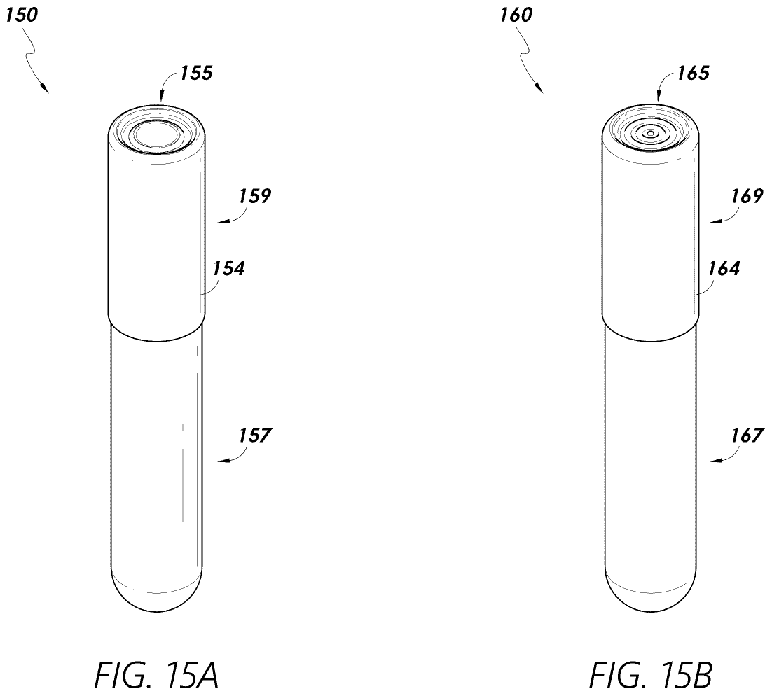

FIG. 15A shows a perspective view of a sensor device 150 having an integrated axial diaphragm 155 in accordance with one or more examples. FIG. 15B show a perspective view of another example sensor device 160 having an integrated axial diaphragm in accordance with one or more examples. The sensor devices 150, 160 in FIGS. 15A and 15B include shell portions/components 159, 169 that are formed of deposition processes in accordance with aspects of the present disclosure, such as physical vapor deposition, or other sputtering technique. The shells 159, 169 are formed in a deposition process to produce a diaphragm 155, 165 that is integrated with the sidewalls/body 154, 164 of the shell 159, 169. Unlike certain other examples disclosed herein, the integrated-diaphragm shells 159, 169 include axially-facing diaphragms 155, 165, which are associated with a distal end/portion of the respective shells 159, 169. The examples of FIGS. 15A and 15B differ in that they include integrated diaphragms that have different structural form. For example, the diaphragm 155 of the device 150 may have a dimpled/speaker-type form, whereas the diaphragm 165 of the device 160 may have a corrugated form comprising multiple concentric rings/corrugations, as described in detail herein. The shells 159, 169, as with other examples disclosed herein, may house/enclose one or more sensor devices/elements. With respect to the axially-facing diaphragms 155, 165, the sensor device(s) disposed within the respective shells 159, 169 may be radially facing or axially facing, according to the particular design implemented.

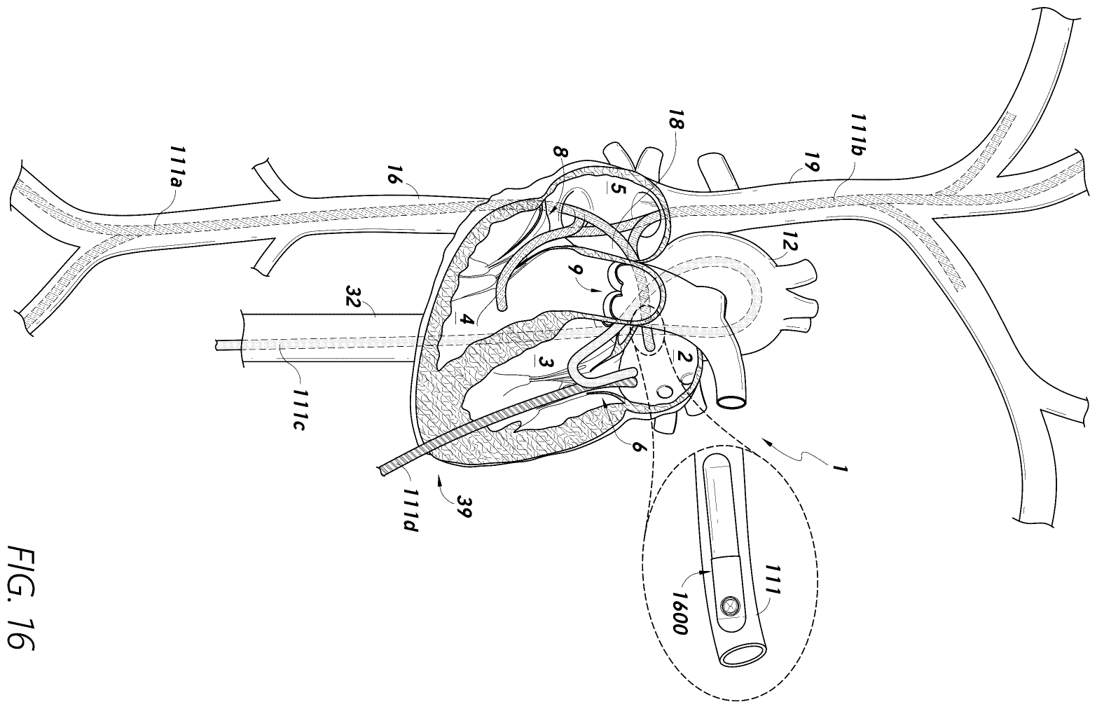

Packaged sensor implant devices in accordance with one or more examples of the present disclosure may be advanced to the relevant target chamber or vessel of the heart and/or vasculature using any suitable or desirable procedure. For example, although access to various chambers/vessels of the heart is illustrated and described in connection with certain examples as being via the right atrium and/or inferior vena cavae, such as through a transfemoral or other transcatheter procedure, other access paths/methods may be implemented in accordance with examples of the present disclosure, as described/shown in connection with FIG. 16. For example, FIG. 16 illustrates various access paths through which access to the chambers of the heart may be achieved. Access to the left atrium or ventricle may be made using transseptal access, which may be made through the inferior vena cava 16 or superior vena cava 19, as respectively shown, and from the right atrium 5, through the septal wall (not shown) and into the left atrium 2. For transaortic access 111c, a delivery catheter may be passed through the descending aorta 32, aortic arch 12, ascending aorta, and aortic valve 7, and into the left atrium 2 through the mitral valve 6. For transapical access 111d, access may be made directly through the apex 39 of the heart into the left ventricle 3, and into the left atrium 2 through the mitral valve 6. Other access paths are also possible beyond those shown in FIG. 16. The various transcatheter delivery systems and paths shown may involve transporting a sensor implant device 1600 within a shaft/lumen of such instrumentation and deploying the device 1600 from the delivery system at the target anatomical site.

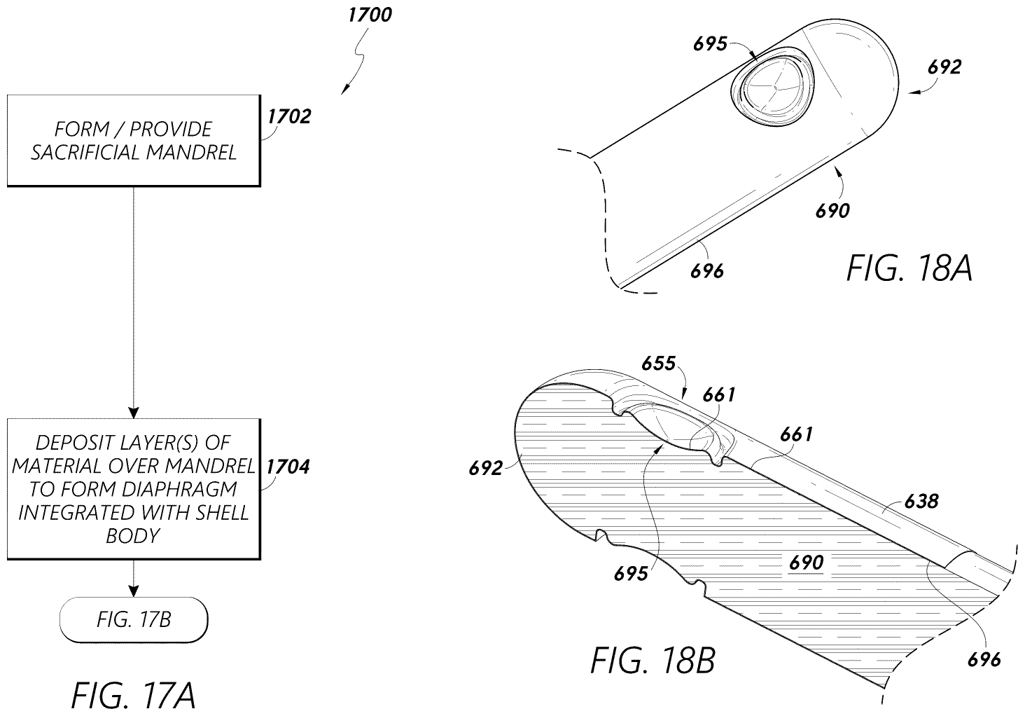

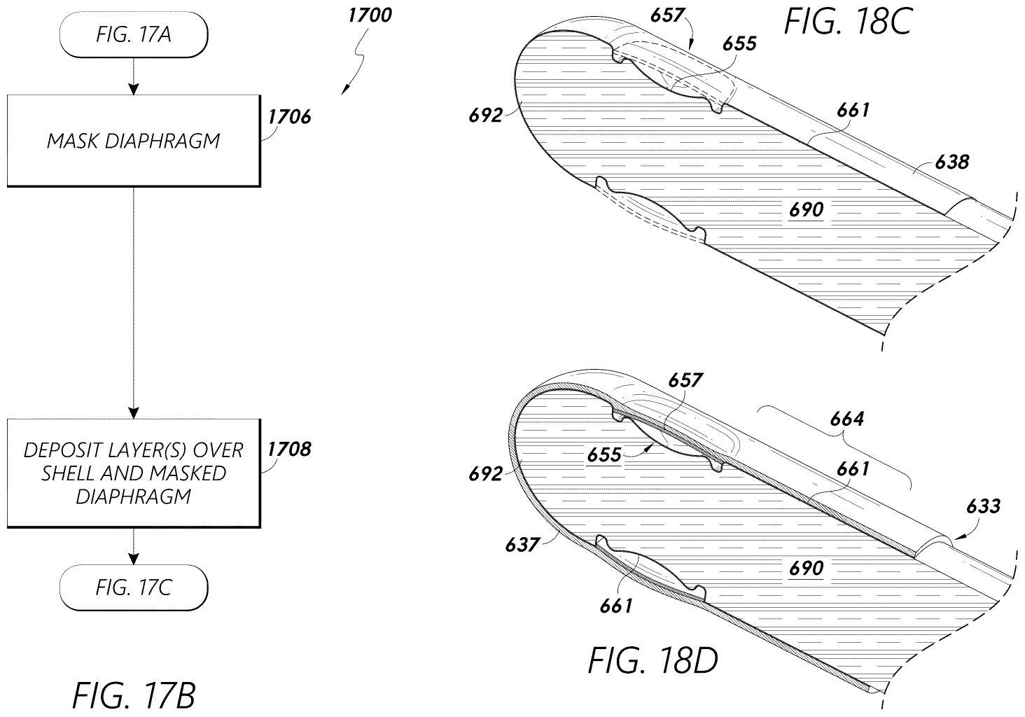

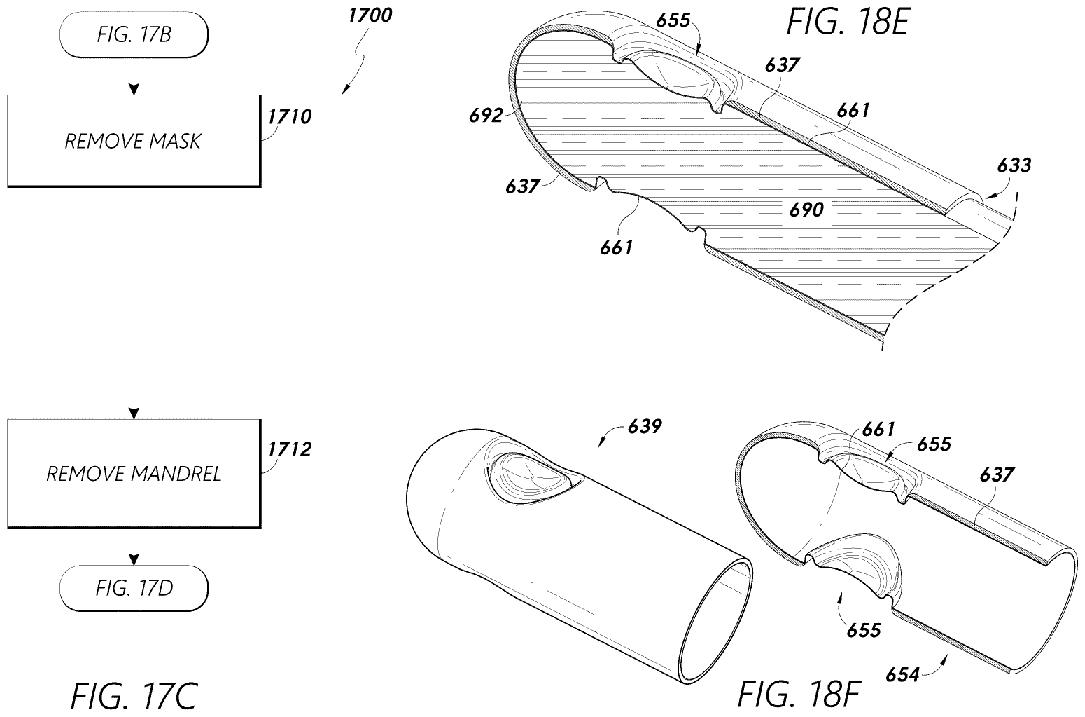

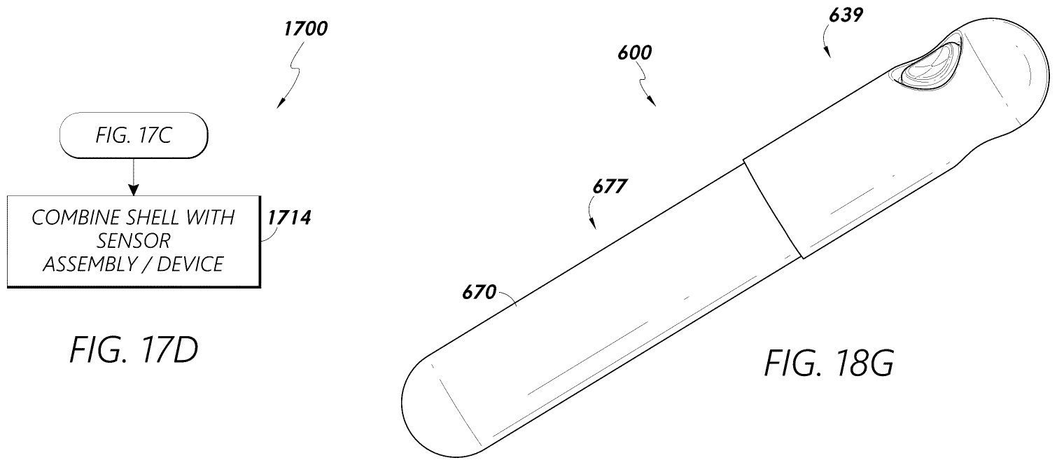

FIGS. 17A-17D provide a flow diagram illustrating a process for packaging a sensor device in accordance with one or more examples. FIGS. 18A-18G provide images of pressure sensor packaging components corresponding to operations of the process of FIGS. 17A-17D according to one or more examples.

At block 1702, the process 1700 involves forming or providing a sacrificial mandrel 690 for formation of a sensor shell as described in detail herein. For example, the sacrificial mandrel 690, shown in image 1802 of FIG. 18A, can comprise material configured to be selectively etched away after deposition on an outer surface thereof of the relevant process material. The mandrel 690 may comprise stainless steel in some examples. The use of an electrically conductive material may be desirable to facilitate direction of deposition vapor in a vacuum chamber, which may be reliant upon implementation of an electric potential between the source material and the mandrel substrate.

The mandrel 690 may be primarily cylindrical in shape, and may be solid or hollow. Examples incorporating hollow mandrels may be advantageous as requiring less sacrificial material etching/removal when deposition is complete. The mandrel 690 may include a dome-shaped and/or flat endcap portion 692. That is, although a semi-sphere dome-type form is shown as the endcap portion 692 in FIG. 18A, it should be understood that the end portion 692 may have any suitable or desirable shape. Furthermore, although the mandrel 690 is shown as having a cylindrical shape, in some implementations, the mandrel (and resulting sensor shell deposition) as a rectangular or elliptical/oval shape.

The mandrel 690 can include diaphragm portion(s) 695 having a shape/form corresponding to a desired pressure diaphragm shape or form, such that deposition of material over such areas can produce the desired diaphragm shape/form for the sensor shell when the conformal deposition is applied to the surface thereof. Although examples are disclosed herein in the context of sensor devices including substantially circular diaphragms, it should be understood that any shape of the diaphragm may be implemented, including elliptical diaphragms.

Although a three-dimensional mandrel form is illustrated and described, wherein the shell is formed by depositing a three-dimensional cylindrical form over the mandrel, it should be understood that examples of the present disclosure may implement two-dimensional deposition of a sheet of material shaped and configured for post-deposition forming of the three-dimensional shell from the sheet of deposited material. Therefore, any example disclosed herein may be understood to comprise a cylindrical sensor shell deposition or deposition of a sheet that is later formed into a sensor shell. However, forming a shell from a sheet of material can introduce complexity in the manufacturing process and complicate the sealing of the shell for biocompatibility purposes.

At block 1704, the process 1700 involves depositing one or more layers 661 of material over the mandrel 690. For example, such layer(s) 661 may be applied over the exterior surface of the mandrel 690 using physical vapor deposition or other deposition or sputtering process, as described in detail above. The deposition material may be any suitable or desirable material compatible with deposition processes as disclosed herein. For example, the deposited layer(s) of material 661 may comprise nitinol, titanium, and/or other metal.

The operation(s) associated with block 1704 may involve using physical vapor deposition to form a shell of material over the sacrificial mandrel 690 and/or over at least a portion thereof. The target material may be applied to the mandrel 690 in one or more layers to create a relatively even deposition of material 661 over the surface of the mandrel 690, such as over substantially the entire surface thereof with respect to one or more perspectives or angles of the mandrel. In some implementations, the initial deposition of layer(s) of shell material 661 is applied over the diaphragm portion 695 of the mandrel 690 to form a diaphragm layer 955, as well as the sidewall 696 and/or end 692 portions. The initial layer(s) 661 applied over the mandrel 690 can be relatively thin, such as, for example, between 10-50 μm thick. FIG. 18B shows the thin shell layer 661 deposited on the mandrel 690.

A particular deposition process implemented may utilize sputtering or evaporation to generate a vapor in the form of molecules or ions from a target source material. Such vapor may be transported and deposited onto the exterior surface of the sacrificial mandrel 690 to create a coating 661. The process of deposition may continue until the desired thickness is achieved. In some implementations, planer magnetron physical vapor deposition is implemented to deposit the layer(s) 661 on the mandrel 690. Such processes can advantageously produce relatively flat application layers. Furthermore, inverted cylindrical magnetron deposition processes can advantageously allow for deposition to occur over a three-dimensional cylindrical surface, as is the mandrel 690 illustrated and described in connection with FIGS. 17 and 18. Deposition layer(s) created by such processes can produce relatively more pristine and isotropic layers compared to wrought forms/materials that are cut, machined, ground, or stamped into the shell form having a diaphragm as described herein.

At block 1706, the process 1700 may involve masking the area of the deposited diaphragm(s) 655 with any suitable or desirable material to cover and protect the diaphragm layer(s) 655 from subsequent deposition(s). The diaphragm mask/cover 657 can be places/disposed over the diaphragm 655 using any type of application process known in the art or described herein. FIG. 18C shows the mask/cover 657 disposed over the diaphragm 655.

In implementations in which the mandrel 690 and shell 638 are removed from the vacuum deposition chamber for the purpose of masking material in the area of the diaphragm 655, wherein further deposition layer(s) are intended for deposition on the shell 638, such removal and/or replacement from/in the vacuum chamber may negatively impact adhesion between the initially deposited layer(s) 661 and any subsequently-deposited layer(s). However, such detrimental effects may nevertheless be desirable as a means of achieving the integrated diaphragm structure with a relatively thin diaphragm as described in connection with the examples disclosed herein. The diaphragm area 655 may be selectively masked in a manner as to avoid application of subsequent layer(s) onto the diaphragm layer(s) 655.

The process 1700 further involves, at block 1708, depositing one or more relatively thick secondary layer(s) 637 over the shell 638 and diaphragm mask(s) 657. The secondary layer(s) 637 of deposition material may be approximately 170-300 μm in thickness, such as between approximately 150-250 μm in some implementations. For example, the thickness may be between about 150-180 μm in some implementations. Such thickness 637 may be combined with the thickness 661 of the end and sidewall portions to form a relatively thick 663 sidewall and endcap structure for the final shell 639, while producing a relatively thinner diaphragm 655, which may be between 3-20% as thick as the combined layer(s) 663 in the sidewall portions. The new deposited(s) 637 can be laid over the mask 657 and the thin layer 661 in the sidewall portions 664, wherein the new/outer layer(s) 637 bond to the outer surface of the thinner shell 661 to create a single/integrated body for the shell 639. The material used for the second deposition 637 associated with block 1708 may advantageously be the same deposition material as applied in the initial deposition process, which may improve adhesion and integration between the layers. The different layers of deposition may be bonded at the molecular level, thereby providing desirable integration. Furthermore, the surfaces of such deposition may be configured to reduce tissue growth and adhesion thereon in some implementations. Although some examples are disclosed in which the primary 661 and secondary 637 deposition layer(s) comprise the same material (e.g., metal or metal alloy), in some implementations, the shell 639 may be formed of primary 661 and secondary 637 depositions that comprise different materials, wherein such layers may comprise materials selected to improve or tune the mechanical properties of the shell 639. FIG. 18D shows the thick layer 637 deposited over the shell 611 (in the area outside of the diaphragm) and the mask 657.

At block 1710, the process 1700 may involve removing the masking/mask 657, thereby revealing the relatively thin diaphragm layer 655 integrated with a relatively thicker shell structure 637. In some implementations, removal of the mask 657 may allow for further deposition in such areas. FIG. 18E shows the mask 657 removed, exposing the thin-layer diaphragm 655.

At block 1712, the process 1700 involves removing the sacrificial mandrel 690. For example, the mandrel substrate (e.g., stainless steel, copper, or the like) may be removed from the completed deposited shell/form 639 to leave the hollow shell. With the internal mandrel etched-out, the complete net form of the shell 639 may be left with a relatively thick structural body 654 and a relatively thinned/thin diaphragm 655. Removing the mandrel can involve dissolving the mandrel form using any suitable or desirable type of etchant. FIG. 18F shows the isolated shell 639 with the mandrel 690 removed.

The isolated shell 639 includes a structural body configured for connection to base component 670 of a sensor device housing, as described herein. The shell 639 includes a tuned thin layer 661 in the diaphragm area 655 to provide desirable pressure transduction functionality, wherein the diaphragm 655 is substantially integrated at the molecular level with the body 654 of the shell 639 without welding, brazing, or other bonding method, which, for reasons described above, can introduce undesired complexity and/or risk of error into the process. The shell 639 is substantially hollow after removal of the mandrel 690.

At block 1712, the process 1700 involves combining the formed shell 639 with the base sensor component 677 which includes a cylindrical (or other shape) open partial capsule 670 configured to house certain electronics, such as an antenna and/or other circuitry for wireless sensor data and/or power transmission. When combined with the base sensor assembly 677, the sensor device 600 may be fully assembled. In some implementations, a sealing or brazing process may be implemented to secure and/or sealed the shell 639 to the housing 670.

The base assembly 677 may be combined with the shell 639, wherein a plug or other structure 645 is disposed at least partially within the base housing 670, wherein such structural feature is configured to hold or secure certain electronic and/or other components within the sensor device 600 and or otherwise provide structural support/integrity for the assembled device 600. The assembled sensor device 600, shown in FIG. 18G, may have any of the features and or characteristics of the sensor device shown in FIGS. 6A-9D.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: An implantable sensor device comprising a cylindrical housing, a deflectable diaphragm associated with a sidewall of the housing, a pressure sensor device housed within the housing, and a transduction medium disposed within the housing over at least a portion of the pressure sensor device.

Example 2: The implantable sensor device of any example herein, in particular example 1, wherein the diaphragm is integrated with the housing.

Example 3: The implantable sensor device of any example herein, in particular example 2, wherein the diaphragm is formed of a layer of deposition material that extends over at least a portion of a sidewall of the housing.

Example 4: The implantable sensor device of any example herein, in particular example 1, wherein the diaphragm lies in a curved plane that is curved around an axis of the housing.

Example 5: The implantable sensor device of any example herein, in particular example 1, wherein the housing comprises a proximal base portion and a distal sensor-enclosing shell portion.

Example 6: The implantable sensor device of any example herein, in particular example 5, wherein the proximal base portion houses an antenna electrically coupled to the pressure sensor device and configured for wireless transmission through the proximal base portion of the housing.

Example 7: The implantable sensor device of any example herein, in particular example 1, wherein the diaphragm comprises a plurality of concentric corrugations.

Example 8: An implantable sensor device comprising a shell formed of a plurality of vacuum deposition metal layers, and a diaphragm formed in a layer of the plurality of metal layers, the layer further forming a body of the shell.

Example 9: The implantable sensor device of any example herein, in particular example 8, wherein the shell has a silo shape including an endcap portion and a cylindrical sidewall portion.

Example 10: The implantable sensor device of any example herein, in particular example 9, wherein the diaphragm is transverse-facing with respect to an axis of the shell and formed in the sidewall portion of the shell.

Example 11: The implantable sensor device of any example herein, in particular example 9, wherein the diaphragm is one of a plurality of diaphragms formed in the sidewall portion of the shell.

Example 12: The implantable sensor device of any example herein, in particular example 11, wherein the plurality of diaphragms comprises at least two circumferentially-distributed diaphragms.

Example 13: The implantable sensor device of any example herein, in particular example 12, wherein the two circumferentially-distributed diaphragms are disposed on opposite circumferential sides of the sidewall portion.

Example 14: The implantable sensor device of any example herein, in particular example 11, wherein the plurality of diaphragms comprises at least two axially-distributed diaphragms.

Example 15: The implantable sensor device of any example herein, in particular example 14, wherein the at least two axially-distributed diaphragms are circumferentially-aligned.

Example 16: The implantable sensor device of any example herein, in particular example 11, wherein the plurality of diaphragms comprises first and second radially-opposite-facing diaphragms.

Example 17: The implantable sensor device of any example herein, in particular example 16, and further comprising a board substrate disposed at least partially within the shell, first and second pressure sensor devices mounted on opposite sides of the board substrate, and a pressure transduction medium disposed between the first and second pressure sensor devices and respective ones of the first and second diaphragms.

Example 18: The implantable sensor device of any example herein, in particular example 8, wherein the diaphragm has an oval shape, wherein a major axis of the oval shape is parallel to an axis of the shell.

Example 19: The implantable sensor device of any example herein, in particular example 8, wherein the shell has a thickness that is at least three times as thick as the diaphragm.

Example 20: A method of manufacturing an implantable sensor device, the method comprising providing a mandrel form, the mandrel form including a body portion including a sidewall portion and an endcap portion and a transverse diaphragm portion on the sidewall portion, placing the mandrel form in a vacuum deposition chamber, depositing one or more base layers of material over at least a portion of the body portion of the mandrel form, and depositing a skin layer over the diaphragm portion and at least a portion of the sidewall portion outside of the diaphragm portion to form a shell including an integrated diaphragm.

Example 21: The method of any example herein, in particular example 20, and further comprising, prior to said depositing the skin layer, removing a portion of the one or more base layers in an area of the diaphragm portion.

Example 22: The method of any example herein, in particular example 21, wherein said removing the portion of the one or more base layers is performed using a laser.

Example 23: The method of any example herein, in particular example 20, and further comprising, prior to said depositing the one or more base layers of material, masking the diaphragm portion.

Example 24: The method of any example herein, in particular example 20, and further comprising, after said depositing the skin layer, removing the mandrel form from the one or more base layers and the skin layer.

Example 25: The method of any example herein, in particular example 24, wherein said removing the mandrel form involves dissolving the mandrel form using an etchant.

Example 26: The method of any example herein, in particular example 20, and further comprising coupling the shell with a base housing having disposed therein an antenna.

Example 27: The method of any example herein, in particular example 20, and further comprising injecting a pressure transduction medium into the shell around a pressure sensor device disposed within the shell.

Example 28: A method of manufacturing an implantable sensor device, the method comprising providing a mandrel form, the mandrel form including a body portion including a sidewall portion and an endcap portion and a transverse diaphragm portion on the sidewall portion, placing the mandrel form in a vacuum deposition chamber, depositing one or more first layers of material over the transverse diaphragm portion and at least a portion of the body portion of the mandrel form to form a diaphragm integrated with a shell body, dispose a mask over the diaphragm, depositing one or more second layers of material over the masked diaphragm and at least a portion of the shell body to form an integrated shell, and removing the mask from over diaphragm.

Example 29: The method of any example herein, in particular example 28, and further comprising, after said depositing the one or more second layers of material, removing the mandrel form from the integrated shell.

Example 30: The method of any example herein, in particular example 29, wherein said removing the mandrel form involves dissolving the mandrel form using an etchant.

Example 31: The method of any example herein, in particular example 28, and further comprising coupling the integrated shell with a base housing having disposed therein an antenna.

Example 32: The method of any example herein, in particular example 28, and further comprising injecting a pressure transduction medium into the integrated shell around a pressure sensor device disposed within the shell.

Example 33: A method of manufacturing an integrated, flexible diaphragm, the method comprising: providing a mandrel form, the mandrel form including a body portion and a diaphragm portion; placing the mandrel form in a vacuum deposition chamber; depositing a plurality of base layers of a material over at least a portion of the body portion of the mandrel form surrounding the diaphragm portion; and depositing a skin layer over the diaphragm portion and over the deposited plurality of base layers around the diaphragm portion to form the integrated, flexible diaphragm.

Example 34: The method of any example herein, especially 33, further comprising prior to said depositing the skin layer, removing a portion of the plurality of base layers in an area of the diaphragm portion.

Example 35: The method of any example herein, especially Example 34, wherein said removing the portion of the plurality of base layers is performed using a laser.

Example 36: The method of any example herein, especially Example 33, further comprising: prior to said depositing the plurality of base layers of material, masking the diaphragm portion with a sacrificial layer.

Example 37: The method of any example herein, especially Example 3, further comprising: after said depositing the skin layer, removing the mandrel form from the one or more base layers and the skin layer.

Example 38: The method of any example herein, especially Example 37, wherein said removing the mandrel form involves dissolving the mandrel form using an etchant.

Example 39: The method of any example herein, especially Example 33, wherein the integrated, flexible diaphragm is flexible relative to the plurality of base layers.

Example 40: The method of any example herein, especially Example 33, wherein the integrated, flexible diaphragm has a thickness that is between 10-40 μm.

Example 41: The method of any example herein, especially Example 33, wherein the integrated, flexible diaphragm has a thickness that is approximately 3-15% of a combined thickness of the skin layer and the plurality of base layers.

Example 42: The method of any example herein, especially Example 33, wherein the integrated, flexible diaphragm comprises a plurality of corrugations.

Example 43: The method of any example herein, especially Example 42, wherein the plurality of corrugations comprise one or more concentric rings.

Example 44: The method of any example herein, especially Example 33, wherein the skin layer comprises a plurality of layers of the material.

Example 45: The method of any example herein, especially Example 33, wherein the skin layer comprises a superelastic nickel titanium material.

Example 46: The method of any example herein, especially Example 45, wherein the material of the plurality of base layers comprises superelastic nickel titanium.

Example 47: The method of any example herein, especially Example 33, wherein the skin layer comprises a superelastic nickel titanium material.

Example 48: The method of any example herein, especially Example 33, wherein the portion of the body portion of the mandrel form surrounding the diaphragm portion is planar.

Example 49: The method of any example herein, especially Example 33, wherein the portion of the body portion of the mandrel form surrounding the diaphragm portion is curved.

Example 50: A method of manufacturing an integrated, flexible diaphragm, the method comprising: providing a mandrel form, the mandrel form including a body portion and a diaphragm portion: placing the mandrel form in a vacuum deposition chamber; depositing one or more first layers of material over the diaphragm portion and at least a portion of the body portion of the mandrel form surrounding the diaphragm portion to form the integrated, flexible diaphragm; disposing a mask layer over the diaphragm; depositing one or more second layers of material over the masked diaphragm and at least the portion of the body portion of the mandrel form surrounding the diaphragm portion.

Example 51: The method of any example herein, especially Example 50, further comprising: prior to said depositing the one or more first layers of material, masking the mandrel form with a sacrificial layer.

Example 52: The method of any example herein, especially Example 50, further comprising: after said depositing the one or more second layers of material, removing the mandrel form from the one or more first layers of material and the one or more second layers of material.

Example 53: The method of any example herein, especially Example 52, wherein said removing the mandrel form involves dissolving the mandrel form or one or more layers thereof using an etchant.

Example 54: The method of any example herein, especially Example 50, wherein the integrated, flexible diaphragm is flexible relative to the one or more second layers of material.

Example 55: The method of any example herein, especially Example 50, wherein the integrated, flexible diaphragm has a thickness that is between 10-40 μm.

Example 56: The method of any example herein, especially Example 50, wherein the integrated, flexible diaphragm has a thickness that is approximately 3-15% of a combined thickness of the one or more first layers of material and the one or more second layers of material.

Example 57: The method of any example herein, especially Example 50, wherein the integrated, flexible diaphragm comprises a plurality of corrugations.

Example 58: The method of any example herein, especially Example 57, wherein the plurality of corrugations comprise one or more concentric rings.

Example 59: The method of any example herein, especially Example 50, wherein the integrated, flexible diaphragm comprises a superelastic nickel titanium material.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having.” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implantable sensor device comprising:
a cylindrical housing;
a deflectable diaphragm associated with a sidewall of the housing;
a pressure sensor device housed within the housing; and
a transduction medium disposed within the housing over at least a portion of the pressure sensor device.

2. The implantable sensor device of claim 1, wherein the diaphragm is integrated with the housing.

3. The implantable sensor device of claim 2, wherein the diaphragm is formed of a layer of deposition material that extends over at least a portion of a sidewall of the housing.

4. The implantable sensor device of claim 1, wherein the diaphragm lies in a curved plane that is curved around an axis of the housing.

5. The implantable sensor device of claim 1, wherein the housing comprises a proximal base portion and a distal sensor-enclosing shell portion.

6. The implantable sensor device of claim 5, wherein the proximal base portion houses an antenna electrically coupled to the pressure sensor device and configured for wireless transmission through the proximal base portion of the housing.

7. The implantable sensor device of claim 1, wherein the diaphragm comprises a plurality of concentric corrugations.

8. An implantable sensor device comprising:
a shell formed of a plurality of vacuum deposition metal layers; and
a diaphragm formed in a layer of the plurality of metal layers, the layer further forming a body of the shell.

9. The implantable sensor device of claim 8, wherein the shell has a silo shape including an endcap portion and a cylindrical sidewall portion.

10. The implantable sensor device of claim 9, wherein the diaphragm is transverse-facing with respect to an axis of the shell and formed in the sidewall portion of the shell.

11. The implantable sensor device of claim 9, wherein the diaphragm is one of a plurality of diaphragms formed in the sidewall portion of the shell.

12. The implantable sensor device of claim 11, wherein the plurality of diaphragms comprises at least two circumferentially-distributed diaphragms.

13. The implantable sensor device of claim 12, wherein the two circumferentially-distributed diaphragms are disposed on opposite circumferential sides of the sidewall portion.

14. The implantable sensor device of claim 11, wherein the plurality of diaphragms comprises at least two axially-distributed diaphragms.

15. The implantable sensor device of claim 14, wherein the at least two axially-distributed diaphragms are circumferentially-aligned.

16. The implantable sensor device of claim 11, wherein the plurality of diaphragms comprises first and second radially-opposite-facing diaphragms.

17. The implantable sensor device of claim 16, and further comprising:
a board substrate disposed at least partially within the shell;
first and second pressure sensor devices mounted on opposite sides of the board substrate; and
a pressure transduction medium disposed between the first and second pressure sensor devices and respective ones of the first and second diaphragms.

18. The implantable sensor device of claim 8, wherein the diaphragm has an oval shape, wherein a major axis of the oval shape is parallel to an axis of the shell.

19. The implantable sensor device of any of claim 8, wherein the shell has a thickness that is at least three times as thick as the diaphragm.

20. A method of manufacturing an implantable sensor device, the method comprising:
providing a mandrel form, the mandrel form including a body portion including a sidewall portion and an endcap portion, and a transverse diaphragm portion on the sidewall portion;
placing the mandrel form in a vacuum deposition chamber;
depositing one or more base layers of material over at least a portion of the body portion of the mandrel form; and
depositing a skin layer over the diaphragm portion and at least a portion of the sidewall portion outside of the diaphragm portion to form a shell including an integrated diaphragm.
